SEQUENCE LISTING (102 sequences)

SEQ ID NO: 1 - protein, 15 aa, synthetic construct
RRPREQIIIGSLWVF

SEQ ID NO: 2 - protein, 15 aa, synthetic construct
IVWHDSLYSTHVQYV

SEQ ID NO: 3 - protein, 15 aa, synthetic construct
NARILQRWFPELAHL

SEQ ID NO: 4 - protein, 15 aa, synthetic construct
SIENYLKKLGYVLLW

SEQ ID NO: 5 - protein, 15 aa, synthetic construct
VIWHPDRWTSVVLQI

SEQ ID NO: 6 - protein, 15 aa, synthetic construct
SVVFSADGRHYYWDS

SEQ ID NO: 7 - protein, 15 aa, synthetic construct
LVFHDNVYTVTVVHV

SEQ ID NO: 8 - protein, 15 aa, synthetic construct
IVLHDGLRSATVLWI

SEQ ID NO: 9 - protein, 15 aa, synthetic construct
RVPRKLVIIGSVWNF

SEQ ID NO: 10 - protein, 15 aa, synthetic construct
LIWYQSYYQTEVVWF

SEQ ID NO: 11 - protein, 15 aa, synthetic construct
IILYRTYFQPFRWEF

SEQ ID NO: 12 - protein, 15 aa, synthetic construct
CFFWYENYYTAITVT

SEQ ID NO: 13 - protein, 15 aa, synthetic construct
TSQICQELDALGDWI

SEQ ID NO: 14 - protein, 15 aa, synthetic construct
ISRWIRQLRVPGFIE

SEQ ID NO: 15 - protein, 15 aa, synthetic construct
DYSLVTVHYWQIVHS

SEQ ID NO: 16 - protein, 15 aa, synthetic construct
ARPREQIIIGSLWVF

SEQ ID NO: 17 - protein, 15 aa, synthetic construct
RAPREQIIIGSLWVF

SEQ ID NO: 18 - protein, 15 aa, synthetic construct
RRAREQIIIGSLWVF

SEQ ID NO: 19 - protein, 15 aa, synthetic construct
RRPAEQIIIGSLWVF

SEQ ID NO: 20 - protein, 15 aa, synthetic construct
RRPRAQIIIGSLWVF

SEQ ID NO: 21 - protein, 15 aa, synthetic construct
RRPREAIIIGSLWVF

SEQ ID NO: 22 - protein, 15 aa, synthetic construct
RRPREQAIIGSLWVF

SEQ ID NO: 23 - protein, 15 aa, synthetic construct
RRPREQIAIGSLWVF

SEQ ID NO: 24 - protein, 15 aa, synthetic construct
RRPREQIIAGSLWVF

SEQ ID NO: 25 - protein, 15 aa, synthetic construct
RRPREQIIIASLWVF

SEQ ID NO: 26 - protein, 15 aa, synthetic construct
RRPREQIIIGALWVF

SEQ ID NO: 27 - protein, 15 aa, synthetic construct
RRPREQIIIGSAWVF

SEQ ID NO: 28 - protein, 15 aa, synthetic construct
RRPREQIIIGSLAVF

SEQ ID NO: 29 - protein, 15 aa, synthetic construct
RRPREQIIIGSLWAF

SEQ ID NO: 30 - protein, 15 aa, synthetic construct
RRPREQIIIGSLWVA

SEQ ID NO: 31 - protein, 18 aa, synthetic construct
YRRPREQIIIGSLWVFCG

SEQ ID NO: 32 - protein, 18 aa, synthetic construct
YIVWHDSLYSTHVQYVCG

SEQ ID NO: 33 - protein, 15 aa, synthetic construct
CRPREQIIIGSLWVF

SEQ ID NO: 34 - protein, 15 aa, synthetic construct
KRPREQIIIGSLWVF

SEQ ID NO: 35 - protein, 15 aa, synthetic construct
RCPREQIIIGSLWVF

SEQ ID NO: 36 - protein, 15 aa, synthetic construct
RRAREQIIIGSLWVF

SEQ ID NO: 37 - protein, 15 aa, synthetic construct
RRLREQIIIGSLWVF

SEQ ID NO: 38 - protein, 15 aa, synthetic construct
RRSREQIIIGSLWVF

SEQ ID NO: 39 - protein, 15 aa, synthetic construct
RRTREQIIIGSLWVF

SEQ ID NO: 40 - protein, 15 aa, synthetic construct
RRNREQIIIGSLWVF

SEQ ID NO: 41 - protein, 15 aa, synthetic construct
RRQREQIIIGSLWVF

SEQ ID NO: 42 - protein, 15 aa, synthetic construct
RRHREQIIIGSLWVF

SEQ ID NO: 43 - protein, 15 aa, synthetic construct
RRKREQIIIGSLWVF

SEQ ID NO: 44 - protein, 15 aa, synthetic construct
RRRREQIIIGSLWVF

SEQ ID NO: 45 - protein, 15 aa, synthetic construct
RRCREQIIIGSLWVF

SEQ ID NO: 46 - protein, 15 aa, synthetic construct
RRPPEQIIIGSLWVF

SEQ ID NO: 47 - protein, 15 aa, synthetic construct
RRPCEQIIIGSLWVF

SEQ ID NO: 48 - protein, 15 aa, synthetic construct
RRPRAQIIIGSLWVF

SEQ ID NO: 49 - protein, 15 aa, synthetic construct
RRPRFQIIIGSLWVF

SEQ ID NO: 50 - protein, 15 aa, synthetic construct
RRPRYQIIIGSLWVF

SEQ ID NO: 51 - protein, 15 aa, synthetic construct
RRPRWQIIIGSLWVF

SEQ ID NO: 52 - protein, 15 aa, synthetic construct
RRPRSQIIIGSLWVF

SEQ ID NO: 53 - protein, 15 aa, synthetic construct
RRPRNQIIIGSLWVF

SEQ ID NO: 54 - protein, 15 aa, synthetic construct
RRPRHQIIIGSLWVF

SEQ ID NO: 55 - protein, 15 aa, synthetic construct
RRPRRQIIIGSLWVF

SEQ ID NO: 56 - protein, 15 aa, synthetic construct
RRPRDQIIIGSLWVF

SEQ ID NO: 57 - protein, 15 aa, synthetic construct
RRPRCQIIIGSLWVF

SEQ ID NO: 58 - protein, 15 aa, synthetic construct
RRPREAIIIGSCWVF

SEQ ID NO: 59 - protein, 15 aa, synthetic construct
RRPREIIIIGSCWVF

SEQ ID NO: 60 - protein, 15 aa, synthetic construct
RRPREYIIIGSCWVF

SEQ ID NO: 61 - protein, 15 aa, synthetic construct
RRPREWIIIGSCWVF

SEQ ID NO: 62 - protein, 15 aa, synthetic construct
RRPRESIIIGSCWVF

SEQ ID NO: 63 - protein, 15 aa, synthetic construct
RRPRETIIIGSCWVF

SEQ ID NO: 64 - protein, 15 aa, synthetic construct
RRPREHIIIGSCWVF

SEQ ID NO: 65 - protein, 15 aa, synthetic construct
RRPREDIIIGSCWVF

SEQ ID NO: 66 - protein, 15 aa, synthetic construct
RRPREEIIIGSCWVF

SEQ ID NO: 67 - protein, 15 aa, synthetic construct
RRPRECIIIGSCWVF

SEQ ID NO: 68 - protein, 15 aa, synthetic construct
RRPREQVIIGSLWVF

SEQ ID NO: 69 - protein, 15 aa, synthetic construct
RRPREQCIIGSLWVF

SEQ ID NO: 70 - protein, 15 aa, synthetic construct
RRPREQIIFGSLWVF

SEQ ID NO: 71 - protein, 15 aa, synthetic construct
RRPREQIIWGSLWVF

SEQ ID NO: 72 - protein, 15 aa, synthetic construct
RRPREQIIICSLWVF

SEQ ID NO: 73 - protein, 15 aa, synthetic construct
RRPREQIIIGCLWVF

SEQ ID NO: 74 - protein, 15 aa, synthetic construct
RRPREQIIIGSVWVF

SEQ ID NO: 75 - protein, 15 aa, synthetic construct
RRPREQIIIGSIWVF

SEQ ID NO: 76 - protein, 15 aa, synthetic construct
RRPREQIIIGSTWVF

SEQ ID NO: 77 - protein, 15 aa, synthetic construct
RRPREQIIIGSRWVF

SEQ ID NO: 78 - protein, 15 aa, synthetic construct
RRPREQIIIGSCWVF

SEQ ID NO: 79 - protein, 15 aa, synthetic construct
RRPREQIIIGSCWGF

SEQ ID NO: 80 - protein, 15 aa, synthetic construct
RRPREQIIIGSCWAF

SEQ ID NO: 81 - protein, 15 aa, synthetic construct
RRPREQIIIGSCWYF

SEQ ID NO: 82 - protein, 15 aa, synthetic construct
RRPREQIIIGSCWSF

SEQ ID NO: 83 - protein, 15 aa, synthetic construct
RRPREQIIIGSCWTF

SEQ ID NO: 84 - protein, 15 aa, synthetic construct
RRPREQIIIGSCWQF

SEQ ID NO: 85 - protein, 15 aa, synthetic construct
RRPREQIIIGSCWHF

SEQ ID NO: 86 - protein, 15 aa, synthetic construct
RRPREQIIIGSCWDF

SEQ ID NO: 87 - protein, 15 aa, synthetic construct
RRPREQIIIGSCWEF

SEQ ID NO: 88 - protein, 15 aa, synthetic construct
RRPREQIIIGSCWCF

SEQ ID NO: 89 - protein, 15 aa, synthetic construct
RRPREQIIIGSCWVA

SEQ ID NO: 90 - protein, 15 aa, synthetic construct
RRPREQIIIGSCWVV

SEQ ID NO: 91 - protein, 15 aa, synthetic construct
RRPREQIIIGSCWVI

SEQ ID NO: 92 - protein, 15 aa, synthetic construct
RRPREQIIIGSCWVL

SEQ ID NO: 93 - protein, 15 aa, synthetic construct
RRPREQIIIGSCWVQ

SEQ ID NO: 94 - protein, 15 aa, synthetic construct
RRPREQIIIGSCWVH

SEQ ID NO: 95 - protein, 15 aa, synthetic construct
RRPREQIIIGSCWVR

SEQ ID NO: 96 - protein, 15 aa, synthetic construct
RRPREQIIIGSCWVE

SEQ ID NO: 97 - protein, 15 aa, synthetic construct
RRPREQIIIGSCWVP

SEQ ID NO: 98 - protein, 15 aa, synthetic construct
RRPREQIIIGSCWVC

SEQ ID NO: 99 - protein, 15 aa, synthetic construct
  VARIANT 3: note = X may be any amino acid.
RRXREQIIIGSLWVF

SEQ ID NO: 100 - protein, 15 aa, synthetic construct
  VARIANT 5: note = X may be any amino acid.
RRPRXQIIIGSLWVF

SEQ ID NO: 101 - protein, 15 aa, synthetic construct
  VARIANT 6: note = X may be any amino acid.
RRPREXIIIGSLWVF

SEQ ID NO: 102 - protein, 15 aa, synthetic construct
  VARIANT 14: note = X may be any amino acid.
RRPREQIIIGSLWXF

(19) United States
(12) Patent Application Publication
(10) Pub. No.: US 2026/0242415 A1
(43) Pub. Date: Aug. 20, 2026

(54) Antiviral cyclic compounds

(71) Applicant: Universiteit Utrecht Holding B.V., Utrecht (NL)

(72) Inventors: Daniel Luke Hurdiss, Houten (NL); Vito Thijssen, Zeist (NL); Franciscus Johannes Maria Van Kuppeveld, Oss (NL); Seino Anne Karel Jongkees, Amsterdam (NL)

(73) Assignee: Universiteit Utrecht Holding B.V., Utrecht (NL)

(21) Appl. No.: 18/876,667

(22) PCT Filed: Jul. 14, 2023
(86) PCT No.: PCT/EP2023/069712

§ 371 Date: Dec. 19, 2024

(30) Foreign Application Priority Data
Jul. 15, 2022 (EP) 22185235.3

(51) Int. Cl. C07K 7/08; A61K 38/00; A61P 31/14

(52) U.S. Cl. CPC C07K 7/08; A61P 31/14; A61K 38/00

(57) ABSTRACT

The present invention relates to novel cyclic peptides. The peptides bind viral proteins, particularly a conserved site on the SARS-CoV-2 spike protein, and therefore the peptides can be useful for neutralizing SARS-CoV-2 variants. Accordingly, the invention also relates to medical use of the peptides.

### FIELD OF THE INVENTION

The present invention relates to novel cyclic peptides. The peptides bind viral proteins, particularly a conserved site on the SARS-CoV-2 spike protein, and therefore the peptides can be useful for neutralizing SARS-CoV-2 variants. Accordingly, the invention also relates to medical use of the peptides.

### BACKGROUND ART

Cellular entry by SARS-CoV-2 is mediated by the membrane spike glycoprotein that recognizes the angiotensin-converting enzyme 2 (ACE2) receptor on the surface of the host cell (Jackson et al., Nat. Rev. Mol. Cell Biol. 23, 3-20, 2022). The spike protein is a trimeric protein that consists of a helical stalk domain (S2) covered by the S1 head on top. The S1 domain is responsible for host-cell receptor binding, whereas S2 facilitates the following membrane fusion. Relevant for infection is proteolytic cleavage at the S1/S2 and S2′ sites by host cell proteases. Collectively, this results in liberation of the S1 unit from the S2 stalk and subsequent tectonic conformational changes in S2 needed to fuse the viral and host membranes. In the prefusion state, each monomer of the S1 domain of the spike protein consists of a receptor-binding domain (RBD) and an N-terminal domain (NTD). The spike protein can exist in either a closed state, in which all RBD are “down” and inaccessible for receptor binding, or an open state, in which at least one of the RBD is in the “up” conformation that is then accessible for ACE2 engagement (Wrapp, D. et al. Science 367, 1260-1263, 2020).

The SARS-CoV-2 spike protein, exposed on the viral membrane, is a promising target for antibodies as a therapeutic intervention for COVID-19. The isolation of human antibodies from convalescent donors has resulted in many potent neutralizing antibodies, of which some are undergoing clinical trials. Most of these antibodies target sites of the RBD that leads to direct interference of ACE2 binding, although different neutralizing sites on the RBD have been identified as well. These SARS-CoV-2 neutralizing antibodies targeting the spike RBD have been classified in literature into 4 classes.

Antibodies from class 1 bind to the receptor-binding motif (RBM) only in the “up” RBD conformation. Class 2 antibodies also bind the RBM on an adjacent site and can bind in both the “up” and “down” state of the RBD. Both classes target sites that have been found to be less conserved between coronaviruses and have also been shown to lose activity against different variants of SARS-CoV-2. Cross-neutralizing antibodies from class 3 and 4 target more conserved sites on the RBD outside the ACE2 binding site, the binding sites of these two have also been described as the S309 proteoglycan site (Class 3) and CR3022 cryptic site (Class 4). The proteoglycan S309 site is accessible in both the “down” and “up” states. Neutralizing antibodies that target this site, like S309 and C135, have been shown to not compete for ACE2 binding and thus have a different mechanism of action. The CR3022 cryptic site, which is located on the opposite side to the S309 site, is named after the SARS-CoV neutralizing antibody CR3022, which was found to also bind the SARS-CoV-2 RBD, although it was non-neutralizing. This site can only be accessed in the ‘up’ conformation of the RBD. As for CR3022, not all antibodies that bind to this site have been found to neutralize viral infection; the ones that do neutralize are proposed to sterically interfere with ACE2 binding based on their solved structure. Although receptor binding happens through interaction with the RBD, neutralizing antibodies targeting the NTD have also been discovered. These antibodies most likely stabilize the prefusion state of the spike, preventing the conformational changes needed for membrane fusion.

Current antibodies that are in clinical trials are heavily affected by the mutations arising in new variants. In a recent study, it was shown that six out of nine antibodies in clinical trials lose their activity against the omicron variant (Planas, D. et al., 2021, doi:10.1101/2021.12.14.472630.) The least affected antibody Sotrovimab, which is derived from S30914, was found to have a 3-fold loss in potency against omicron. Two other antibodies, Cilgavimab (Class 2) and Adintrevimab (Epitope in between Class 1 and 4), showed a 20-fold decrease in activity. This demonstrates the difficulty in antibody development against mutating viruses and the need to target conserved epitopes.

Production of antibodies is laborious, and their storage, transport, and administration to subjects are sensitive to various factors. More stable, easier to manufacture, and cost-efficient alternatives would be very attractive.

As a result, there is a need for improved treatment, prevention, or amelioration of viral infections. There is a need for antiviral substances with improved stability. There is a need for antiviral substances with a more robust activity profile. There is a need for substances that can neutralize SARS-CoV-2 through new mechanisms of action, e.g. through modulation of the conformational dynamics of a viral spike protein. There is a need for substances that can modulate the conformational dynamics of a viral spike protein.

### SUMMARY OF THE INVENTION

The invention provides a compound of general formula (I):

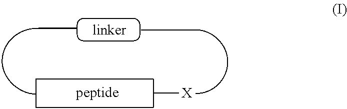

wherein peptide is an oligopeptide having 13-17 amino acid residues, wherein the first amino acid is preferably arginine, lysine, histidine, leucine, isoleucine, or valine; the seventh amino acid is preferably isoleucine, leucine, or valine; and the twelfth amino acid is preferably isoleucine, leucine, or valine; linker is a linking moiety that together with X connects the N-terminus and the C-terminus of peptide to form a macrocyclic structure; and X is O or NH, or a salt of such a compound.

In preferred embodiments, linker comprises 1 to 12 optionally substituted backbone atoms selected from carbon, nitrogen, oxygen, and sulphur, wherein optional substitutions can be ═O, halogen, C1-4 hydrocarbon, C1-4 acyl, C1-4 alkoxy, —C(═O)-oligopeptide, —SH, —S—(C1-4 hydrocarbon), —NH₂, —NH—(C1-4 hydrocarbon), —NH—(C1-4 acyl), —N—(C1-4 hydrocarbon)₂, —N—(C1-4 acyl)₂, an amino acid side chain, or a targeting moiety. Preferably linker has general formula (L1):

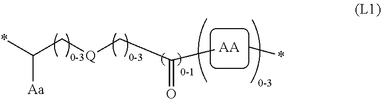

wherein Aa is H, —NH₂, —COOH, —CONH₂, -(AA)ₙ-X′, —C(═O)(AA)ₙ-X′, or a dimerizing linker; Q is CH₂, O, S, or NH; and AA is in each instance an independently selected amino acid residue; X′ is OH or NH₂; and n is 1, 2, 3, 4, 5, 6, 7, 8, 9, or 10. In more preferred embodiments linker has general formula (L2):

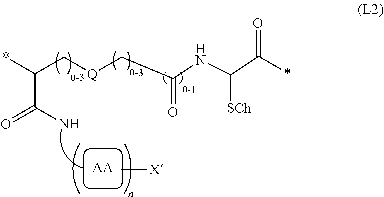

wherein X′ is OH or NH₂; Q is CH₂, O, S, or NH; SCh is an amino acid side chain; AA is in each instance an independently selected amino acid residue; and n is 1, 2, 3, 4, or 5. Even more preferably linker has general formula (L3):

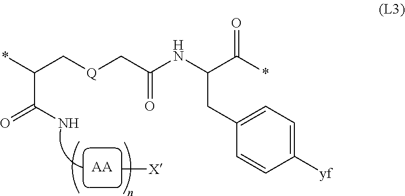

wherein X′ is OH or NH₂; Q is CH₂, O, S, or NH; yf is H or OH; AA is in each instance an independently selected amino acid residue; and n is 1, 2, or 3. Highly preferred are embodiments wherein linker has a general formula selected from (L4a) through (L4h) as depicted later herein.

Preferably peptide is an oligopeptide having 13-17 amino acid residues, wherein the first amino acid is arginine, histidine, or lysine, preferably arginine; the seventh amino acid is isoleucine or leucine, preferably isoleucine; the twelfth amino acid is isoleucine or leucine, preferably leucine. In some embodiments peptide comprises a sequence represented by any one of SEQ ID NOs: 1-30, wherein up to six positions can be substituted by another amino acid. Preferably peptide has 15 amino acid residues; and/or peptide comprises a sequence represented by any one of SEQ ID NOs: 1-2; and/or linker has any one of general formulas (L4a) through (L4h); and/or X is NH. In preferred embodiments peptide has a length of 15-17, preferably 15 amino acids and is an oligopeptide represented by SEQ ID NO: 1 having 5, 4, 3, 2, or 1 amino acid substitutions.

In a highly preferred embodiment the compound is cyclic peptide 1 or a salt thereof:

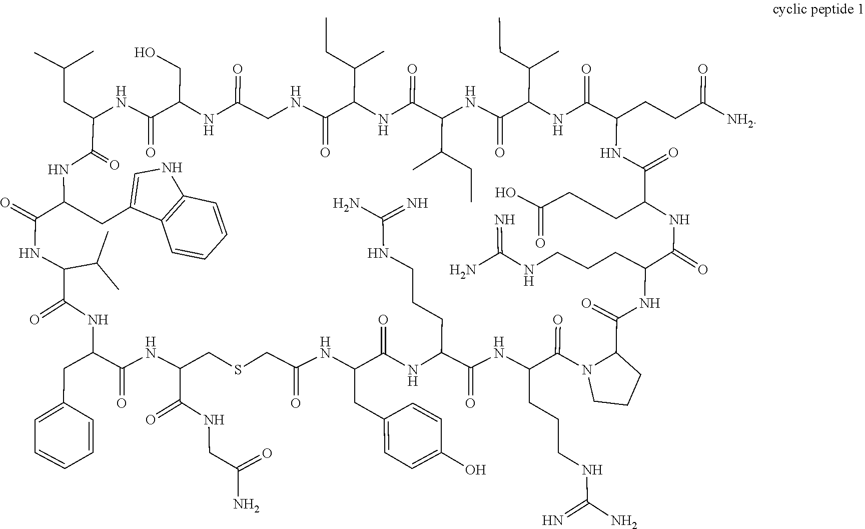 [SMILES: CCC(C)C1NC(=O)C(CCC(N)=O)NC(=O)C(CCC(=O)O)NC(=O)C(CCCNC(=N)N)NC(=O)C2CCCN2C(=O)C(CCCNC(=N)N)NC(=O)C(CCCNC(=N)N)NC(=O)C(Cc2ccc(O)cc2)NC(=O)CSCC(C(=O)NCC(N)=O)NC(=O)C(Cc2ccccc2)NC(=O)C(C(C)C)NC(=O)C(Cc2c[nH]c3ccccc23)NC(=O)C(CC(C)C)NC(=O)C(CO)NC(=O)CNC(=O)C(C(C)CC)NC(=O)C(C(C)CC)NC1=O]

Also provided is a composition comprising a compound as defined above and a pharmaceutically acceptable excipient. Also provided is the compound or the composition for use as a medicament. Preferably the medicament is for treating a viral infection, preferably a coronaviral infection, more preferably a SARS-CoV-2 infection.

Also provided is an in vitro, in vivo, or ex vivo method for modulating the conformational dynamics of a viral spike protein, wherein the method comprises the step of contacting the viral spike protein with a compound or composition as defined above. Also provided as is a method of treating a viral infection, the method comprising administering to a subject a compound or composition as defined above.

### DESCRIPTION OF EMBODIMENTS

The inventors have found that several oligopeptides have a high affinity for viral proteins. The identity of some residues within these oligopeptides was found to be of great influence on protein binding. Accordingly, the invention provides a compound of general formula (I):

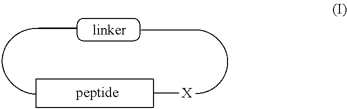

wherein
peptide is an oligopeptide having 13-17 amino acid residues, wherein
   the first amino acid is preferably arginine, lysine, histidine, leucine, isoleucine, or valine;
   the seventh amino acid is preferably isoleucine, leucine, or valine; and
   the twelfth amino acid is preferably isoleucine, leucine, or valine;
linker is a linking moiety that together with X connects the N-terminus and the C-terminus of peptide to form a macrocyclic structure; and
X is O or NH, or a salt of such a compound. Such a compound is referred to herein as a compound according to the invention. In some embodiments X is O. In preferred embodiments X is NH.

A salt of a compound according to the invention is preferably a pharmaceutically acceptable salt. Such salts include salts derived from inorganic bases such as Li, Na, K, Ca, Mg, Fe, Cu, Zn and Mn; salts of organic bases such as N,N′-diacetylethylenediamine, glucamine, triethylamine, choline, dicyclohexylamine, benzylamine, trialkylamine, thiamine, guanidine, diethanolamine, alpha-phenylethylamine, piperidine, morpholine, pyridine, hydroxyethylpyrrolidine, hydroxyethylpiperidine, and the like. Such salts also include amino acid salts such as glycine, alanine, cystine, cysteine, lysine, arginine, phenylalanine, guanidine, etc. Such salts may include acid addition salts where appropriate, which are for example sulphates, nitrates, phosphates, perchlorates, borates, hydrohalides such as HCl or HBr salts, acetates, trifluoroacetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Preferred salts are HCl salts, formic acid salts, acetic acid salts, and trifluoroacetic acid salts. More preferred salts are HCl salts and trifluoroacetic acid salts, most preferably HCl salts.

The compound according to the invention can be a hydrate or a solvate. In the context of the invention a hydrate refers to a solvate wherein the solvent is water. The term solvate, as used herein, refers to a crystal form of a substance which contains solvent. Solvates are preferably pharmaceutically acceptable solvates and may be hydrates or may comprise other solvents of crystallization such as alcohols, ether, and the like.

### Peptide Moiety

peptide is an oligopeptide having 13-17 amino acid residues, wherein the first amino acid is preferably arginine, lysine, histidine, leucine, isoleucine, or valine; the seventh amino acid is preferably isoleucine, leucine, or valine; and the twelfth amino acids is preferably isoleucine, leucine, or valine. In preferred embodiments peptide is an oligopeptide having 13-17 amino acid residues, wherein the first amino acid is arginine, lysine, histidine, leucine, isoleucine, or valine; the seventh amino acid is isoleucine, leucine, or valine; and the twelfth amino acids is isoleucine, leucine, or valine.

Amino acid residues as comprised in peptide can be any amino acids, not limited to naturally occurring proteinogenic amino acids. Herein, the use of the term oligopeptide should be so construed that next to oligopeptides where each residue is a naturally occurring proteinogenic amino acid linked to its neighbor through a backbone amide bond, also peptides comprising non-natural amino acids, peptidomimetics, unconventional linkages, and other variations are encompassed. This includes oligopeptides comprising alkylated bonds, inverted bonds, or other types of bonds, such as esters, triazoles, carbamates, ureas, thioureas, imides, imines, halogenated bonds, alpha-halogenated bonds, ketones, or oligopeptides comprising beta-amino acids, other extended amino acids, or peptoids where side chains of residues are attached to backbone amide bonds instead of to the corresponding alpha carbon atoms.

The term ‘amino acid’ as used herein should be interpreted as any moiety that can constitute a residue in a polypeptide as defined above. peptide can comprise amino acids of any chirality, such as L-amino acids or D-amino acids, or mixtures thereof. Most often, an amino acid is a molecular acid, preferably featuring a carboxylic acid, said amino acid featuring an amine at an alpha-carbon next to the carboxylic acid. However, the amine can also be more distant from the carboxylic acid. The most common naturally occurring proteinogenic amino acids and their three-letter abbreviations and one-letter codes are the following: Alanine (Ala, A); Arginine (Arg, R), Asparagine (Asn, N); Aspartic acid (Asp, D); Cysteine (Cys, C); Glutamic acid (Glu, E); Glutamine (Gln, Q); Glycine (Gly, G); Histidine (His, H); Isoleucine (Ile, I); Leucine (Leu, L); Lysine (Lys, K); Methionine (Met, M); Phenylalanine (Phe, F); Proline (Pro, P); Serine (Ser, S); Threonine (Thr, T); Tyrosine (Tyr, Y); Tryptophan (Trp, W); Valine (Val, V). Later herein, where indicated, X is additionally used for L-3,4-di-hydroxy-phenylalanine, and Z is additionally used for pentafluorophenylalanine. Naturally occurring amino acids are also called natural amino acids. Natural amino acids are often proteinogenic, which means that they are used by organisms in the biosynthesis of proteins. In some cases, natural amino acids can also be non-proteinogenic. Natural amino acids are those amino acids that can be found in nature, without further limiting their role or function.

It is to be understood that amino acids as comprised in peptide are radicals, wherein at least one hydrogen of an amine is absent, and one hydroxyl moiety of a carboxylic acid moiety is absent. This is in line with the three-letter representation of for instance H-Gly-OH, where the first H represents an H on the amine of glycine, and the OH represents the hydroxyl part of the carboxylic acid of glycine. In this context, X can be seen as replacing the H— or —OH of a terminal amino acid of peptide as comprised in a compound according to the invention. Similarly, linker replaces the H— or —OH of the other terminal amino acid of peptide. Preferably, X replaces —OH and linker replaces H—. In other words, preferably X is C-terminal and linker is N-terminal. It should be understood that as used throughout this document, a terminus can indicate the end of a moiety and does not necessarily indicate a terminal end of the entire compound. In fact, compounds according to the invention are generally macrocyclic and could be said to not comprise an actual terminus.

In preferred embodiments, peptide is an oligopeptide having 13-17 amino acid residues wherein the amino acid residues are L-amino acids or D-amino acids or a mixture thereof. More preferably, peptide is an oligopeptide having 13-17 amino acid residues wherein the amino acid residues are L-amino acids. Preferably, peptide is an oligopeptide having 13-17 amino acid residues wherein the amino acid residues are naturally occurring proteinogenic amino acids or pentafluorophenylalanine or L-DOPA. Preferably, peptide is an oligopeptide having 13-17 amino acid residues wherein the amino acid residues are naturally occurring proteinogenic amino acids. More preferably, peptide is an oligopeptide having 13-17 amino acid residues wherein the amino acid residues are L-amino acids or D-amino acids or a mixture thereof and are naturally occurring proteinogenic amino acids. Most preferably, peptide is an oligopeptide having 13-17 amino acid residues wherein the amino acid residues are L-amino acids and are naturally occurring proteinogenic amino acids.

Amino acids are often characterized by the nature of their side chains. Amino acids that are considered to be basic amino acids are lysine, arginine, and histidine. Amino acids that are considered to be acidic amino acids are aspartic acid, glutamic acid, and tyrosine. Amino acids that are considered to be polar uncharged amino acids are serine, threonine, cysteine, asparagine, and glutamine. Amino acids that are considered to be hydrophobic amino acids are alanine, valine, isoleucine, leucine, methionine, phenylalanine, tyrosine, proline, and tryptophan. Proline is considered to be a conformationally restrained amino acid. Glycine is achiral yet can be considered both a D-amino acid and an L-amino acid. Additional moieties can be conjugated to the side chains of some amino acid residues, such as protecting groups, alkyl groups such as C1-6 alkyl groups, or additional amino acid residues such as glycine.

Within the embodiments of this invention, peptide can be comprised in a larger oligopeptide, for instance when linker comprises further amino acid residues. Oligopeptides are further understood to possibly feature protecting groups such as t-butyl carbamate, 9-fluorenylmethyl carbamate, benzyl carbamate, benzyl ester, t-butyl ester, methyl ester, or other protecting groups. Preferably, amino acid residues are not protected in compounds according to the invention.

peptide has 13-17 amino acid residues. Thus peptide has 13, 14, 15, 16, or 17 amino acid residues. These amino acid residues are the amino acid residues forming the backbone of the oligopeptide. Additional amino acid residues may be present when for instance linked to the side chain of amino acid residues that are comprised in the backbone of the oligopeptide. In some embodiments, peptide has 13 amino acid residues. In some embodiments, peptide has 14 amino acid residues. In some embodiments, peptide has 15 amino acid residues. In some embodiments, peptide has 16 amino acid residues. In some embodiments, peptide has 17 amino acid residues.

In some embodiments peptide has 13-16 amino acid residues. In some embodiments peptide has 13-15 amino acid residues. In some embodiments peptide has 13-14 amino acid residues. In some embodiments peptide has 14-17 amino acid residues. In some embodiments peptide has 14-16 amino acid residues. In some embodiments peptide has 14-15 amino acid residues. In some embodiments peptide has 15-17 amino acid residues. In some embodiments peptide has 15-16 amino acid residues. In some embodiments peptide has 16-17 amino acid residues. Preferably peptide has 14-16 amino acid residues, more preferably 14-15 amino acid residues, most preferably 15 amino acid residues.

Systematic experimental studies have revealed that certain amino acid residues strongly contribute to the binding properties of the compound according to the invention. Good properties were found when the first amino acid is arginine, lysine, histidine, leucine, isoleucine, or valine; the seventh amino acid is isoleucine, leucine, or valine; and the twelfth amino acid is isoleucine, leucine, or valine. In counting the amino acid residues, the count is preferably started at the position opposite of X. This X is as depicted in general formula (I). In highly preferred embodiments the count is started at the N-terminus of peptide. The count is consecutive.

In preferred embodiments peptide is an oligopeptide having 13-17 amino acid residues, wherein the first amino acid is arginine, histidine, or lysine, preferably arginine; the seventh amino acid is isoleucine or leucine, preferably isoleucine; the twelfth amino acids is isoleucine or leucine, preferably leucine.

In some preferred embodiments the first amino acid is arginine, lysine, or histidine, most preferably arginine. In some preferred embodiments the first amino acid is leucine, isoleucine, or valine, most preferably isoleucine. In highly preferred embodiments the first amino acid is arginine or isoleucine.

In some preferred embodiments the seventh amino acid is isoleucine. In some preferred embodiments the seventh amino acid is valine. In highly preferred embodiments the seventh amino acid is isoleucine or valine.

In some preferred embodiments the twelfth amino acid is leucine. In some preferred embodiments the twelfth amino acid is valine. In highly preferred embodiments, the twelfth amino acid is leucine or valine.

When the first amino acid is arginine, lysine, or histidine, the seventh amino acid is preferably isoleucine or leucine, more preferably isoleucine. When the first amino acid is arginine, lysine, or histidine, the twelfth amino acid is preferably isoleucine or leucine, more preferably leucine.

When the first amino acid is leucine, isoleucine, or valine, the seventh amino acid is preferably isoleucine or leucine, more preferably leucine. When the first amino acid is leucine, isoleucine, or valine, the twelfth amino acid is preferably valine.

Herein, when an oligopeptide is provided, it can be procured from a commercial source or obtained by isolating it from a natural product. Oligopeptides can be obtained through isolation from a digest of a larger protein. Preferably, oligopeptides are of synthetic origin. A preferred method for oligopeptide synthesis is solid-phase peptide synthesis (SPPS), which is well-known to a person skilled in the art. Advantages of obtaining short peptides through SPPS are the ease of synthesis, the low component cost, the speed of synthesis, and the possibility for automation using synthesis robots, synthesizers, semi-automatic synthesizers, or automatic synthesizers. SPPS strategies known in the art allow both N-terminal and C-terminal modification, such as alkylation, amidation, or labeling. A person skilled in the art will understand that when an amino acid is referred to as having a certain characteristic, reference is generally being made to the side chain of said amino acid. As a non-limiting example, when phenylalanine is referred to as a hydrophobic amino acid, its amine and its carboxylic acid moieties are not taken into consideration.

In preferred embodiments peptide comprises a sequence represented by any one of SEQ ID NOs: 1-30, wherein up to six positions can be substituted by another amino acid. Preferably peptide does not comprise further amino acid residues. In some embodiments, up to five positions can be substituted by another amino acid, wherein substitutions preferably comprise substitution at residues selected from 4, 5, 6, 9, 14, and 15, more preferably 4, 5, 6, 14, and 15. Systematic study has revealed that these positions of the compounds according to the invention are very tolerant to mutation. In some embodiments, up to five positions can be substituted by another amino acid, wherein substitutions preferably comprise substitution at residues 14 and/or 15, more preferably at both residues 14 and 15.

In some embodiments, up to four positions can be substituted by another amino acid, wherein substitutions preferably comprise substitution at residues selected from 4, 5, 6, 14, and 15. In some embodiments, up to four positions can be substituted by another amino acid, wherein substitutions preferably comprise substitution at residues 14 and/or 15, more preferably at both residues 14 and 15.

In some embodiments, up to three positions can be substituted by another amino acid, wherein substitutions preferably comprise substitution at residues selected from 4, 5, 6, 14, and 15. In some embodiments, up to three positions can be substituted by another amino acid, wherein substitutions preferably comprise substitution at residues 14 and/or 15, more preferably at both residues 14 and 15.

In some embodiments, up to two positions can be substituted by another amino acid, wherein substitutions preferably comprise substitution at residues selected from 4, 5, 6, 14, and 15. In some embodiments, up to two positions can be substituted by another amino acid, wherein substitutions preferably comprise substitution at residues 14 and/or 15, more preferably at both residues 14 and 15.

In some embodiments, one position can be substituted by another amino acid, wherein the substitution preferably is at a residue selected from 4, 5, 6, 14, and 15, preferably selected from 14 and 15. Suitable SEQ ID NOs for peptide are shown below. The sequences are artificial.

The following substitutions are referred to herein as the preferred substitutions. In these preferred substitutions, one letter amino acid codes are used, with the additional definitions of Z referring to pentafluorophenylalanine (2-Amino-3-(pentafluorophenyl) propanoic acid) and X referring to L-DOPA (L-3,4-di-hydroxy-phenylalanine):

When position 1 is substituted by another amino acid, it is preferably substituted by Z, Q, L, R, or C, more preferably by Q, K, or C, most preferably K.

When position 2 is substituted by another amino acid, it is preferably substituted by Z, R, or C, more preferably by C.

When position 3 is substituted by another amino acid, it is preferably substituted by A, L, Z, X, S, T, N, Q, H, L, R, P, or C, more preferably by L, S, T, N, K, R, or C, most preferably by C.

When position 4 is substituted by another amino acid, it is preferably substituted by A, Z, R, P, or C, more preferably by Z, P, or C, most preferably by C.

When position 5 is substituted by another amino acid, it is preferably substituted by A, F, Y, X, W, S, N, H, R, D, E, or C, more preferably by A, Y, X, W, S, N, H, or C, most preferably by X, W, or C.

When position 6 is substituted by another amino acid, it is preferably substituted by A, I, Y, X, W, S, T, Q, H, D, E, or C, more preferably by X, W, E, or C, most preferably by C.

When position 7 is substituted by another amino acid, it is preferably substituted by V or I, most preferably by V.

When position 8 is substituted by another amino acid, it is preferably substituted by I.

When position 9 is substituted by another amino acid, it is preferably substituted by I, F, Z, or W, more preferably by Z or W, most preferably by Z.

When position 10 is substituted by another amino acid, it is preferably substituted by G.

When position 11 is substituted by another amino acid, it is preferably substituted by S or C.

When position 12 is substituted by another amino acid, it is preferably substituted by V, I, L, Z, T, R, or C, more preferably by V, I, Z, or R, most preferably by V or I.

When position 13 is substituted by another amino acid, it is preferably substituted by W.

When position 14 is substituted by another amino acid, it is preferably substituted by G, A, V, Y, X, S, T, Q, H, D, E, or C, more preferably by G, A, S, T, Q, H, D, E, or C, even more preferably by G, H, D, or E, most preferably by E.

When position 15 is substituted by another amino acid, it is preferably substituted by A, V, I, L, F, Z, Q, H, R, E, P, or C, more preferably by A, V, I, L, Q, E, or P, even more preferably by I, L, Q, or E, most preferably by I or L.

The above preferred substitutions are preferably substitutions in SEQ ID NO: 1. In highly preferred embodiments peptide is an oligopeptide having 15-17 amino acids, preferably having 15 amino acids, and comprising SEQ ID NO: 1 wherein the above preferred substitutions can be made, preferably at most 5 of the above preferred substitutions can be made, more preferably at most 4, still more preferably at most 3, even more preferably at most 2, most preferably only one of the above preferred substitutions can be made.

In preferred embodiments peptide is an oligopeptide represented by SEQ ID NO: 1 and the above preferred substitutions are made only on positions 2, 3, 4, 5, 6, 7, 9, 12, 14, or 15, more preferably only in positions 2, 3, 5, 6, 7, 12, 14, or 15, still more preferably only in positions 5, 12, or 14, most preferably only on positions 12 or 14.

In highly preferred embodiments peptide is an oligopeptide represented by SEQ ID NO: 1 and the above preferred substitutions are made only on positions 2, 3, 4, 5, 6, 7, 9, 12, 14, or 15, more preferably only in positions 2, 3, 5, 6, 7, 12, 14, or 15, still more preferably only in positions 5, 12, or 14, most preferably only on positions 12 or 14.

In highly preferred embodiments peptide has a length of 15-17, preferably 15 amino acids and is an oligopeptide represented by SEQ ID NO: 1 having 5, 4, 3, 2, or 1 substitutions, wherein when position 1 is substituted by another amino acid, it is preferably substituted by Z, Q, L, or C, more preferably by Q, K, or C, most preferably K; when position 2 is substituted by another amino acid, it is preferably substituted by Z or C, more preferably by C; when position 3 is substituted by another amino acid, it is preferably substituted by A, L, Z, X, S, T, N, Q, H, L, R, or C, more preferably by L, S, T, N, K, R, or C, most preferably by C; when position 4 is substituted by another amino acid, it is preferably substituted by A, Z, P, or C, more preferably by Z, P, or C, most preferably by C; when position 5 is substituted by another amino acid, it is preferably substituted by A, F, Y, X, W, S, N, H, R, D, or C, more preferably by A, Y, X, W, S, N, H, or C, most preferably by X, W, or C; when position 6 is substituted by another amino acid, it is preferably substituted by A, I, Y, X, W, S, T, H, D, E, or C, more preferably by X, W, E, or C, most preferably by C; when position 7 is substituted by another amino acid, it is preferably substituted by V; position 8 is not substituted; when position 9 is substituted by another amino acid, it is preferably substituted by F, Z, or W, more preferably by Z or W, most preferably by Z; when position 10 is substituted by another amino acid, it is preferably substituted by C; when position 11 is substituted by another amino acid, it is preferably substituted by C; when position 12 is substituted by another amino acid, it is preferably substituted by V, I, Z, T, R, or C, more preferably by V, I, Z, or R, most preferably by V or I; position 13 is not substituted; when position 14 is substituted by another amino acid, it is preferably substituted by G, A, Y, X, S, T, Q, H, D, E, or C, more preferably by G, A, S, T, Q, H, D, E, or C, even more preferably by G, H, D, or E, most preferably by E; when position 15 is substituted by another amino acid, it is preferably substituted by A, V, I, L, Z, Q, H, R, E, P, or C, more preferably by A, V, I, L, Q, E, or P, even more preferably by I, L, Q, or E, most preferably by I or L; wherein substitutions are preferably made only on positions 2, 3, 4, 5, 6, 7, 9, 12, 14, or 15, more preferably only in positions 2, 3, 5, 6, 7, 12, 14, or 15, still more preferably only in positions 5, 12, or 14, most preferably only on positions 12 or 14.

| SEQ ID NO: | Sequence | SEQ ID NO: | |
|---|---|---|---|
| 1 | RRPREQIIIGSLWVF | 17 | RAPREQIIIGSLWVF |
| 2 | IVWHDSLYSTHVQYV | 18 | RRAREQIIIGSLWVF |
| 3 | NARILQRWFPELAHL | 19 | RRPAEQIIIGSLWVF |
| 4 | SIENYLKKLGYVLLW | 20 | RRPRAQIIIGSLWVF |
| 5 | VIWHPDRWTSVVLQI | 21 | RRPREAIIIGSLWVF |
| 6 | SVVFSADGRHYYWDS | 22 | RRPREQAIIGSLWVF |
| 7 | LVFHDNVYTVTVVHV | 23 | RRPREQIAIGSLWVF |
| 8 | IVLHDGLRSATVLWI | 24 | RRPREQIIAGSLWVF |
| 9 | RVPRKLVIIGSVWNF | 25 | RRPREQIIIASLWVF |
| 10 | LIWYQSYYQTEVVWF | 26 | RRPREQIIIGALWVF |
| 11 | IILYRTYFQPFRWEF | 27 | RRPREQIIIGSAWVF |
| 12 | CFFWYENYYTAITVT | 28 | RRPREQIIIGSLAVF |
| 13 | TSQICQELDALGDWI | 29 | RRPREQIIIGSLWAF |
| 14 | ISRWIRQLRVPGFIE | 30 | RRPREQIIIGSLWVA |
| 15 | DYSLVTVHYWQIVHS | 31 | YRRPREQIIIGSLWVFCG |
| 16 | ARPREQIIIGSLWVF | 32 | YIVWHDSLYSTHVQYVCG |

Preferred are SEQ ID NOs: 1-13 and 16-32 and preferably also 33-102, more preferably SEQ ID NOs: 1-13 and 16-30 and preferably also 33-102. More preferred are SEQ ID NOs: 1-13, 17-21, 23-26, and 28-30 and preferably also 33-102. In some embodiments they are SEQ ID NOs: 1-13 and preferably also 33-102. In some embodiments they are SEQ ID NOs: 1, 17-21, 23-26, and 28-30. In some embodiments they are SEQ ID NOs: 1 and 2 and preferably also 33-102. SEQ ID NO: 1 is most preferred and preferably also 33-102.

The invention provides an oligopeptide comprising or consisting of the amino acid residues of peptide as defined above. Accordingly, the invention provides a compound represented by H-peptide-OH. Accordingly, the invention provides an oligopeptide comprising or consisting of the sequence represented by any one of SEQ ID NOs: 1-30. More preferred SEQ ID NOs are SEQ ID NOs: 1-13, 17-21, 23-26, and 28-30. In some embodiments the SEQ ID NOs are SEQ ID NOs: 1-13. In some embodiments the SEQ ID NOs are SEQ ID NOs: 1, 17-21, 23-26, and 28-30. In some embodiments the SEQ ID NOs are SEQ ID NOs: 1 and 2. SEQ ID NO: 1 is most preferred. The invention also provides an oligonucleotide comprising a sequence that encodes an oligopeptide comprising or consisting of the sequence represented by any one of SEQ ID NOs: 1-32, preferably 1-13 and 16-32. These are preferably peptides consisting of the sequence represented by the SEQ ID NOs which are acetylated at the N-terminus and amidated at the C-terminus.

Preferred compound are those wherein peptide is an oligopeptide represented by any one of SEQ ID NOs: 1, 33-102, more preferably those denote +, ++, +++, or ++++ in Table S4, even more preferably those denoted ++, +++, or ++++, still more preferably those denoted +++ or ++++, most preferably those denoted ++++, being SEQ ID NOs: 74 or 87.

### Linker Moiety

linker is a linking moiety that together with X connects the N-terminus and the C-terminus of peptide to form a macrocyclic structure. This macrocyclic structure is thus formed by the backbone of peptide, by X, and by linker.

Preferably, linker comprises 1 to 12 optionally substituted backbone atoms selected from carbon, nitrogen, oxygen, and sulphur, wherein optional substitutions can be ═O, halogen, C1-4 hydrocarbon, C1-4 acyl, C1-4 alkoxy, —C(═O)-oligopeptide, —SH, —S—(C1-4 hydrocarbon), —NH₂, —NH—(C1-4 hydrocarbon), —NH—(C1-4 acyl), —N—(C1-4 hydrocarbon)₂, —N—(C1-4 acyl)₂, an amino acid side chain, or a targeting moiety.

As used here, backbone atoms are the atoms that form the macrocycle. This is in analogy with the backbone atoms of a macrocyclic peptide. Preferably linker comprises 2 to 12, more preferably 3 to 12 backbone atoms, even more preferably 4 to 11, 5 to 10, 6 to 9, or 7 to 9 backbone atoms. Most preferably linker comprises 8 backbone atoms. In some embodiments the backbone atoms are not substituted.

In preferred embodiments the backbone atoms are substituted. Amongst others, the optional substitutions can be ═O, halogen, C1-4 hydrocarbon, C1-4 acyl, C1-4 alkoxy, —C(═O)-oligopeptide, —SH, —S—(C1-4 hydrocarbon), —NH₂, —NH—(C1-4 hydrocarbon), —NH—(C1-4 acyl), —N—(C1-4 hydrocarbon)₂, —N—(C1-4 acyl)₂, an amino acid side chain, or a targeting moiety. Preferably they can be ═O, —C(═O)-oligopeptide, an amino acid side chain, or a targeting moiety, more preferably ═O, —C(═O)-oligopeptide, and an amino acid side chain.

C1-4hydrocarbon is preferably acyl, alkyl, cycloalkyl, or heterocycloalkyl. Each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, trifluoromethyl, or optionally interrupted by one or more heteroatoms. A skilled person will understand that the valency of atoms is always to be fulfilled. In this context, heterocycloalkyl is to be interpreted as cycloalkyl that has been interrupted by one or more heteroatoms. Acyl moieties are alkyl moieties wherein the proximal carbon atom is substituted by an oxo moiety (═O). Alkoxy moieties are —O-alkyl moieties. In this context, haloalkyl is to be interpreted as alkyl that has been substituted with halogen. A preferred haloalkyl is a fluorinated alkyl, more preferably a perfluorinated alkyl, most preferably trifluoromethyl. In the context of the invention, halogen is fluorine (F), chlorine (Cl), bromine (Br), or iodine (I). Preferred halogens are fluorine, chlorine, and bromine, more preferred halogens are fluorine or chlorine, a most preferred halogen is fluorine.

In the context of this invention, the number of carbon atoms in a moiety such as alkyl, acyl, cycloalkyl, heterocycloalkyl, is indicated as for example C1-4, in this non-limiting case indicating that from 1 to 4 carbon atoms are envisaged, such as 1, 2, 3, or 4 carbon atoms. Similarly C2-4alkyl has 2, 3, or 4 carbon atoms. The number of carbon atoms can be expressed as the total number of carbon atoms not counting further substitutions, the total number of carbon atoms, or as the number of carbon atoms that can be found in the longest continuous internal sequence of carbon atoms. Preferably, the number of carbon atoms is expressed as the total number of carbon atoms not counting further substitutions.

In the context of this invention, unsubstituted alkyl groups have the general formula CₙH₂ₙ₊₁ and may be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of suitable alkyl groups to, include, but are not limited-CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, and the like. Preferred alkyl groups are linear or branched, most preferably, linear. Cycloalkyl groups are cyclic alkyl groups; preferred cycloalkyl groups are cyclopropyl and cyclobutyl. Heterocycloalkyl groups are cycloalkyl groups N. Preferred C₁₋₄alkyl groups are —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, cyclopropyl, and cyclobutyl, more preferably, —CH₃, —CH₂CH₃, I CH₂CH₂CH₃, —CH(CH₃)₂, —CH(CH₃)CH₂CH₃, —CH₂CH(CH₃)₂, —CH₂CH₂CH₂CH₃, and —C(CH₃)₃.

Alkyl groups of the invention are optionally unsaturated. In preferred embodiments, alkyl is not unsaturated. Unsaturated alkyl groups are preferably alkenyl or alkynyl groups. In the context of this invention, unsubstituted alkenyl groups have the general formula CₙH₂ₙ₋₁, and may be linear or branched. Examples of suitable alkenyl groups include, but are not limited to, ethenyl, propenyl, isopropenyl, butenyl, pentenyl and the like. Unsubstituted alkenyl groups may also contain a cyclic moiety, and thus have the concomitant general formula CₙH₂ₙ₋₃. Preferred alkenyl groups are linear or branched, most preferably, linear. Highly preferred unsaturated cycloalkyl groups are aryl groups, such as phenyl.

Unsubstituted alkynyl groups have the general formula CₙH₂ₙ₋₃ and may be linear or branched. Unsubstituted alkynyl groups may also contain a cyclic moiety, and thus have the concomitant general formula CₙH₂ₙ₋₅. Optionally, the alkynyl groups are substituted by one or more substituents further specified in this document. Examples of suitable alkynyl groups include, but are not limited to, ethynyl, propargyl, n-but-2-ynyl, n-but-3-ynyl, and octyne such as cyclooctyne. Preferred alkyl groups are linear or branched, most preferably linear.

Aryl groups are aromatic and generally comprise at least six carbon atoms and may include monocyclic, bicyclic and polycyclic structures. Optionally, the aryl groups may be substituted by one or more substituents further specified in this document. Examples of aryl groups include groups such as phenyl, naphthyl, anthracyl and the like. A heteroaryl group is aromatic and comprises one to four heteroatoms selected from the group consisting of S, O, and N. Due to the heteroatoms it can have a smaller ring size than six.

In this invention, each instance of alkyl, acyl, cycloalkyl, and heterocycloalkyl is optionally substituted, preferably with one or more moieties selected from halogen, oxy, hydroxyl, methyl, ethyl, propyl, methoxy, ethoxy, trifluoromethyl, wherein each instance can also be interrupted by a heteroatom such as N, O, or S, and wherein each instance of alkyl, acyl, alkoxyl, cyclyl, and heterocyclyl is optionally unsaturated. Interruption by a heteroatom means interruption by one or more heteroatoms. In this context, preferably no more than 20, more preferably 1, 2, 3, 4, or 5 heteroatoms interrupt, even more preferably 1, 2, or 3, preferably 1 or 2, most preferably 1 heteroatom interrupts. Preferably all interrupting heteroatoms are of the same element. As a non-limiting example, the C₅alkyl-CH₂—CH₂—CH₂—CH₂—CH₃ when interrupted by heteroatoms can be —CH₂—CH₂—O—CH₂—CH₂—O—CH₃. In preferred embodiments, there is no optional substitution. In preferred embodiments, there is both substitution and unsaturation.

In preferred embodiments, C1-4alkyl when optionally unsaturated and optionally substituted can be C1-4alkyl, C₁-4acyl, C2-4alkenyl, C₂-4alkynyl, C3-4cycloalkyl, C₃-4heterocycloalkyl, optionally substituted with one or more moieties selected from halogen, oxy, hydroxyl, methyl, ethyl, propyl, methoxy, ethoxy, and trifluoromethyl. In preferred embodiments, C1-4alkyl when optionally unsaturated and optionally substituted can be C1-4alkyl, C₁-4acyl, C2-4alkenyl, C₂-4alkynyl, C3-4cycloalkyl, or C3-4heterocycloalkyl, optionally substituted with one or more moieties selected from halogen, oxy, hydroxyl, methyl, ethyl, propyl, methoxy, ethoxy, and trifluoromethyl.

—C(═O)-oligopeptide can comprise any oligopeptide, but it is preferably short, such as comprising only 4, 3, 2, or 1 amino acid. The —C(═O) moiety of —C(═O)-oligopeptide can be the C-terminus of the oligopeptide, but it can also be an additional carbonyl moiety that is comprised in an amide bond together with the N-terminus of the oligopeptide. The latter is preferred. The oligopeptide can have a free terminus but is preferably amidated. Preferred oligopeptides for —C(═O)-oligopeptide comprise up to three amino acids chosen from alanine and glycine, more preferably glycine, even more preferably one or two amino acids, most preferably one amino acid such as glycine. A highly preferred —C(═O)-oligopeptide is —C(═O)—NH—CH₂—C(═O)—NH₂.

An amino acid side chain can be any amino acid side chain known in the art. Preferably it can for each instance independently be H or optionally substituted and optionally unsaturated C2-6(halo)alkyl-NH₂, C1-6(halo)alkyl, 5-10-membered (hetero)aryl, C1-4(halo)alkyl-[5-10-membered (hetero)aryl], C2-6(halo)alkyl-N(H)C(NH₂)(═NH), C1-6(halo)alkyl-C(O)—NH₂, or C1-4(halo)alkyl-[3-10-membered (hetero)cycloalkyl]; in some embodiments an amino acid side chain is not optionally substituted and not optionally unsaturated; in some embodiments an amino acid side chain is optionally substituted and not optionally unsaturated; in some embodiments an amino acid side chain is not optionally substituted and is optionally unsaturated; particularly preferred optional substitutions are halogen, C1-3(halo)alkyl, or C1-3(halo)alkoxyl, more preferably halogen, —CH₃, and —O—CH₃; wherein within an amino acid side chain optional substitutions are preferably —OH, —SH, —SeH, —S—CH₃, —O—CH₃, and —COOH, more preferably —OH, —SH, and —S—CH₃, most preferably —OH; an amino acid side chain is preferably H, C2-5(halo)alkyl-NH₂, C1-4(halo)alkyl, 5-9-membered (hetero)aryl, C1-2(halo)alkyl-[5-9-membered (hetero)aryl], C2-4(halo)alkyl-N(H)C(NH₂)(═NH), C1-4(halo)alkyl-C(O)—NH₂, or C1-2(halo)alkyl-[3-9-membered (hetero)cycloalkyl]; wherein C2-6(halo)alkyl-NH₂ is preferably —CH₂—CH₂—CH₂—NH₂ or —CH₂—CH₂—CH₂—CH₂—NH₂; wherein 5-10-membered (hetero)aryl is preferably phenyl; wherein optionally substituted C1-4(halo)alkyl-[5-10-membered (hetero)aryl] is preferably —CH₂-phenyl, —CH₂—CH₂-phenyl, —CH₂-imidazolyl, —CH₂—CH₂-imidazolyl, —CH₂-indolyl, —CH₂—CH₂-indolyl; —CH₂-hydroxyphenyl, —CH₂—CH₂-hydroxyphenyl; wherein optionally substituted C1-6(halo)alkyl is preferably —CH₃, —CH(CH₃)₂, —CH₂—CH(CH₃)₂, —CH(CH₃)—CH₂—CH₃, —CH₂—OH, —CH₂—SH, —CH₂—SeH, —CH₂—CH₂—CH₂—S—CH₃, —CH(CH₃)—CH₂—OH, —CH₂—CH₂—COOH), or —CH₂—COOH), more preferably —CH₃, —CH(CH₃)₂, —CH₂—CH(CH₃)₂, —CH(CH₃)—CH₂—CH₃, —CH₂—OH, —CH₂—SH, —CH₂—SeH, —CH₂—CH₂—CH₂—S—CH₃, or —CH₂(CH₃)—CH₂—OH; wherein C2-6(halo)alkyl-N(H)C(NH₂)(═NH) is preferably —CH₂—CH₂—CH₂—N—C(═NH)—NH₂; wherein C1-6(halo)alkyl-C(O)—NH₂ is preferably —CH₂—CH₂—C(O)NH₂ or —CH₂—C(O)NH₂.

A targeting moiety can be any moiety that is known to target or bind a target of interest. Examples of targeting moieties are cell-penetrating peptides, receptor ligands, and antibodies or fragments thereof. A compound according to the invention is also a targeting moiety because it binds spike proteins. This means that compounds of general formula (I) can be dimers, linked via linker. A targeting moiety is preferably connected to the remainder of linker via a linker, wherein this linker preferably comprises one or more amino acids, oligo (ethylene glycol), or a C2-12 hydrocarbon. A preferred targeting moiety is a dimerizing linker. More features of a dimerizing linker are provided below. A dimer that is formed as such thus comprises two macrocycles. Preferably, peptide and X are identical for both macrocycles. Preferably, linker is identical for both macrocycles.

In preferred embodiments, linker has general formula (L1):

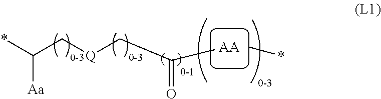

wherein
Aa is H, —NH₂, —COOH, —CONH₂, -(AA)ₙ-X′, —C(═O)(AA)ₙ-X′, or a dimerizing linker; preferably Aa is H, —COOH, —C(═O)(AA)ₙ-X′, or a dimerizing linker, more preferably it is-COOH, —C(═O)(AA)ₙ-X′, or a dimerizing linker, still more preferably it is —COOH or —C(═O)(AA)ₙ-X′, most preferably —C(═O)(AA)ₙ-X′;
Q is CH₂, O, S, or NH; preferably, Q is S or NH, more preferably it is S; and
AA is in each instance an independently selected amino acid residue; preferably AA is glycine, alanine, phenylalanine, or tyrosine, most preferably glycine or tyrosine; when comprised in -(AA)ₙ-X′ or —C(═O)(AA)ₙ-X′, it is preferably glycine or alanine, most preferably glycine; when not comprised in -(AA)ₙ-X′ or —C(═O)(AA)ₙ-X′, it is preferably phenylalanine or tyrosine, more preferably tyrosine;
X′ is OH or NH₂; preferably it is NH₂; and
n is 1, 2, 3, 4, 5, 6, 7, 8, 9, or 10; preferably it is 1, 2, 3, 4, 5, or 6, more preferably 1, 2, 3, or 4, more preferably 1, 2, or 3, even more preferably 1 or 2, most preferably it is 1.

Preferably, linker has general formula (L2):

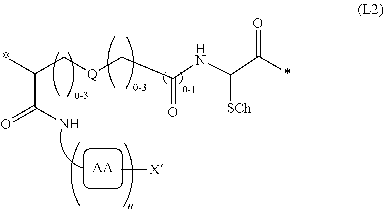

wherein
X′ is OH or NH₂, preferably NH₂;
Q is CH₂, O, S, or NH, preferably S;
SCh is an amino acid side chain, preferably it is -(phenyl) or -(phenyl)-OH, more preferably (phenyl)-OH, most preferably in the para configuration;
AA is in each instance an independently selected amino acid residue; preferably selected from glycine and alanine, more preferably glycine; and
n is 1, 2, 3, 4, or 5, preferably 1, 2, 3, or 4, more preferably 1, 2, or 3, most preferably 1.

In some embodiments, linker has general formula (L3):

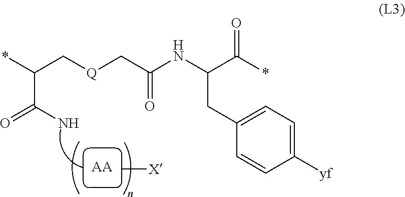

wherein
X′ is OH or NH₂; preferably NH₂;
Q is CH₂, O, S, or NH; preferably S;
yf is H or OH; preferably OH;
AA is in each instance an independently selected amino acid residue; preferably selected from glycine and alanine, more preferably glycine; and
n is 1, 2, or 3, preferably 1 or 2, most preferably 1.

Preferred examples of linker are those wherein linker has a general formula selected from (L4a) through (L4h):

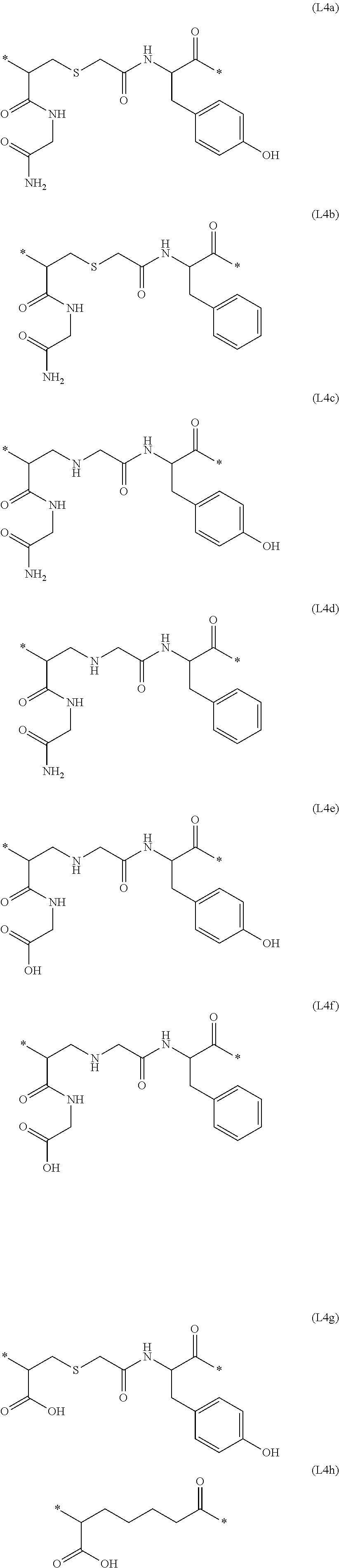

(L4a) through (L4g) are more preferred, (L4a) and (L4b) are even more preferred, (L4a) is most preferred. In preferred embodiments, chirality for the above moieties, where present, is as shown for the moieties as comprised in Dimer 1.

The dimerizing linker is to connect two otherwise individual compounds of general formula (I), resulting in a compound comprising two macrocycles. The nature of the dimerizing linker is not important as long as it connects the two macrocycles to one another. A skilled person knows how to select a dimerizing linker, and can form dimers out of individual macrocycles as described herein. A general formula for a dimer can be seen as general formula (Dimer1):

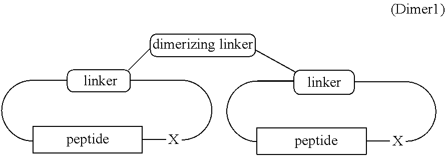

For ease of representation, when dimerizing linker is schematically represented, it is generally represented herein as a mere shape comprising the text “dimerizing linker”, wherein the dimerizing linker represents both the linker and the second macrocycle, as shown in (dimer1). Suitable instances of linker to use in compounds of general formula (dimer1) are shown below. In preferred embodiments, chirality for the below moieties, where present, is as shown for the moieties as comprised in Dimer 1. Of the below, DiL4 is most preferred:

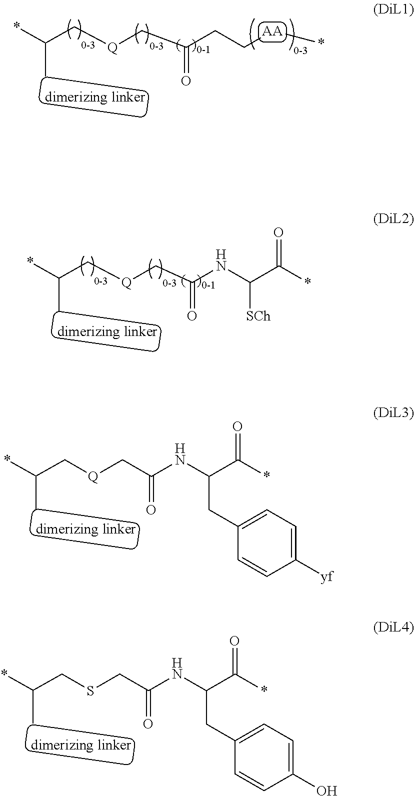

The dimerizing linker itself, connecting the two macrocycles, can be selected by a skilled person. It preferably has a backbone length of 6-120 atoms and is optionally substituted, more preferably of 10-80 atoms, even more preferably of 15-70 atoms, still more preferably 20-60 atoms, still more preferably 30-55 atoms, more preferably of 40-55 atoms, most preferably of 45-54 atoms, such as 50, 51, or 52 atoms, of which 51 atoms is particularly preferred. For convenience of their synthesis, the atoms are preferably short oligoethylene glycol repeats, or short alkyl chains, optionally connected via amide, urea, or carbamate links. The optional substitutions are generally for ease of synthesis, and are preferably ═O, halogen, C1-4 hydrocarbon, C1-4 acyl, C1-4 alkoxy, —C(═O)-oligopeptide, —SH, —S—(C1-4 hydrocarbon), —NH₂, —NH—(C1-4 hydrocarbon), —NH—(C1-4 acyl), —N—(C1-4 hydrocarbon)₂, —N—(C1-4 acyl)₂, or an amino acid side chain, all as described above; more preferably they are ═O, —C(═O)-oligopeptide, or an amino acid side chain, most preferably —C(═O)-oligopeptide such as —C(═O)—NH—CH₂—C(═O)—NH₂. Preferred dimerizing linkers are as follows:

| | |
|---|---|
| DL1 | —C(═O)NHCH₂C(═O)[NH(CH₂CH₂O)₂CH₂C(═O)]₂NH(CH₂)₄C[—C(═O)—NH—CH₂—C(═O)— |
| | NH₂]NH[C(═O)CH(OCH₂CH₂)₂NH]₂C(═O)CH₂NHC(═O)— |
| DL2 | —C(═O)NHCH₂C(═O)[NH(CH₂CH₂O)₂CH₂C(═O)]₂NH(CH₂)₄C[—C(═O)— |
| | OH]NH[C(═O)CH(OCH₂CH₂)₂NH]₂C(═O)CH₂NHC(═O)— |
| DL3 | —C(═O)NHCH₂C(═O)[NH(CH₂CH₂O)₂CH₂C(═O)]₂NH(CH₂)₄C[—C(═O)— |
| | NH₂]NH[C(═O)CH(OCH₂CH₂)₂NH]₂C(═O)CH₂NHC(═O)— |
| DL4 | —C(═O)NHCH₂C(═O)[NH(CH₂CH₂O)₂CH₂C(═O)]₃NH(CH₂)₄C[—C(═O)—NH—CH₂—C(═O)— |
| | NH₂]NH[C(═O)CH(OCH₂CH₂)₂NH]₃C(═O)CH₂NHC(═O)— |
| DL5 | —C(═O)NHCH₂C(═O)[NH(CH₂CH₂O)₂CH₂C(═O)]₄NH(CH₂)₄C[—C(═O)—NH—CH₂—C(═O)— |
| | NH₂]NH[C(═O)CH(OCH₂CH₂)₂NH]₄C(═O)CH₂NHC(═O)— |
| DL6 | —C(═O)NHCH₂C(═O)[NH(CH₂CH₂O)₂CH₂C(═O)]NH(CH₂)₄C[—C(═O)—NH—CH₂—C(═O)— |
| | NH₂]NH[C(═O)CH(OCH₂CH₂)₂NH]C(═O)CH₂NHC(═O)— |
| DL7 | —C(═O)NHCH₂C(═O)[NH(CH₂CH₂O)₂CH₂C(═O)]₆NH(CH₂)₄C[—C(═O)—NH—CH₂—C(═O)— |
| | NH₂]NH[C(═O)CH(OCH₂CH₂)₂NH]₆C(═O)CH₂NHC(═O)— |
| DL8 | —C(═O)NHCH₂C(═O)[NH(CH₂CH₂O)₂CH₂C(═O)]₈NH(CH₂)₄C[—C(═O)—NH—CH₂—C(═O)— |
| | NH₂]NH[C(═O)CH(OCH₂CH₂)₂NH]₈C(═O)CH₂NHC(═O)— |
| DL9 | —C(═O)NHCH₂C(═O)[NH(CH₂CH₂O)₂CH₂C(═O)]₁₀NH(CH₂)₄C[—C(═O)—NH—CH₂—C(═O)— |
| | NH₂]NH[C(═O)CH(OCH₂CH₂)₂NH]₁₀C(═O)CH₂NHC(═O)— |

In some embodiments it is DL1, DL2, or DL3. In some embodiments it is DL1, DL4-DL9. In some embodiments it is DL1 or DL4. In some embodiments it is DL1, DL5, DL6, or DL8. Highly preferred is DL5. A preferred compound according to the invention comprises linker that is DiL4 wherein dimerizing linker is DL5. It is preferred for such a compound that peptide comprises SEQ ID NO: 1. Such a dimer is preferred, and such an embodiment can be represented by Dimer 1:

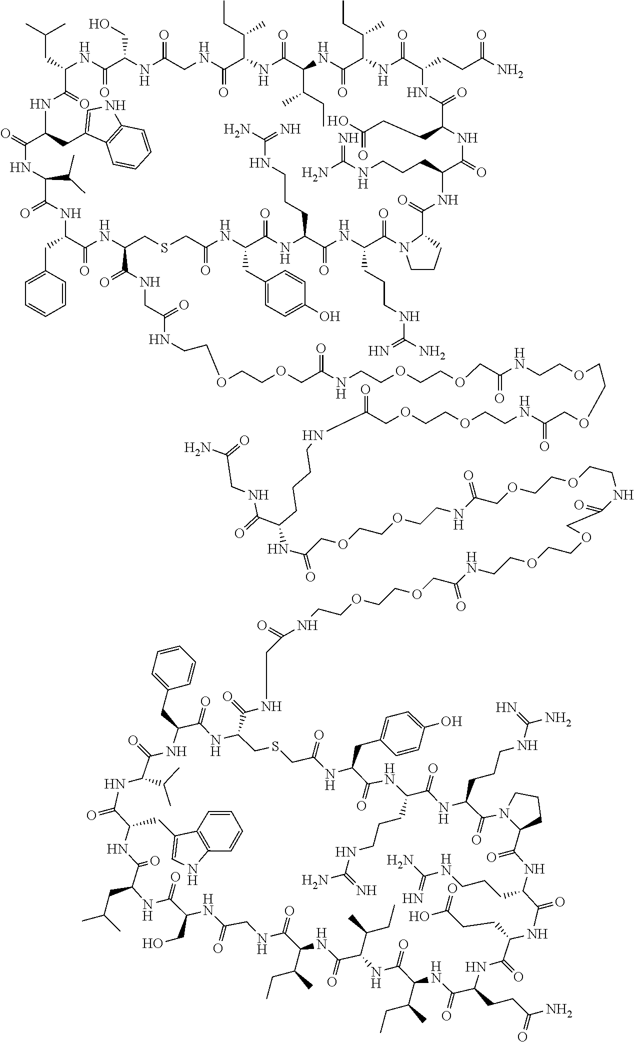

### Further Definitions of the Compound

In preferred embodiments,
peptide has 15 amino acid residues; and/or
peptide comprises a sequence represented by any one of SEQ ID NOs: 1-2; and/or
linker has any one of general formulas (L4a) through (L4h); and/or
X is NH.

Preferably peptide has 15 amino acid residues; and peptide comprises a sequence represented by any one of SEQ ID NOs: 1-2. Preferably peptide has 15 amino acid residues and peptide comprises a sequence represented by any one of SEQ ID NOs: 1-2 X is NH. Preferably peptide has 15 amino acid residues; and peptide comprises a sequence represented by any one of SEQ ID NOs: 1-2; and linker has any one of general formulas (L4a) through (L4h). Preferably peptide has 15 amino acid residues; and peptide comprises a sequence represented by any one of SEQ ID NOs: 1-2; and linker has any one of general formulas (L4a) through (L4h); and X is NH.

In preferred embodiments, the compound of general formula (I) is cyclic peptide 1 or a salt thereof:

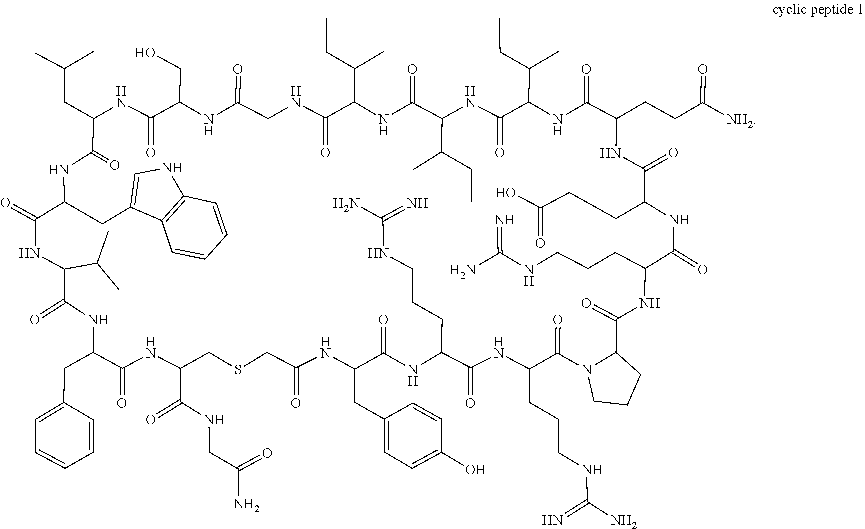

A preferred cyclic peptide 1 is cyclic peptide 1L:

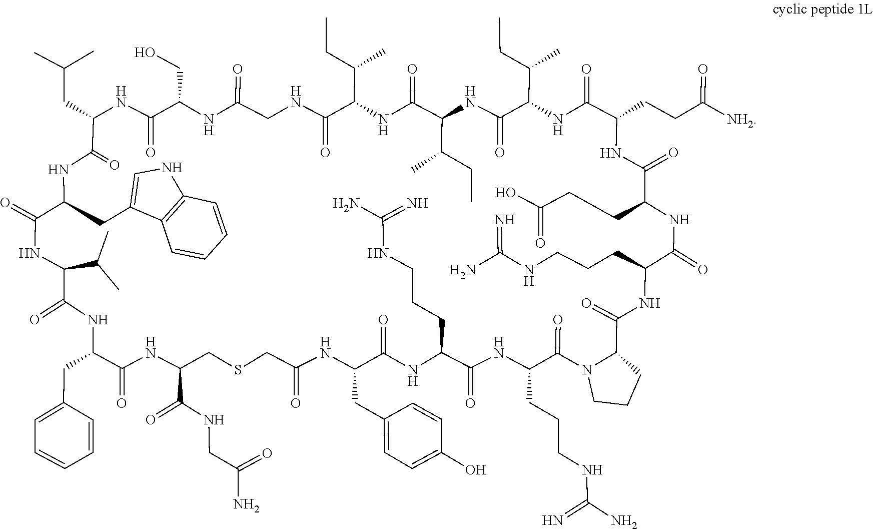 [SMILES: CC[C@H](C)[C@@H]1NC(=O)[C@H](CCC(N)=O)NC(=O)[C@H](CCC(=O)O)NC(=O)[C@H](CCCNC(=N)N)NC(=O)[C@@H]2CCCN2C(=O)[C@H](CCCNC(=N)N)NC(=O)[C@H](CCCNC(=N)N)NC(=O)[C@H](Cc2ccc(O)cc2)NC(=O)CSC[C@@H](C(=O)NCC(N)=O)NC(=O)[C@H](Cc2ccccc2)NC(=O)[C@H](C(C)C)NC(=O)[C@H](Cc2c[nH]c3ccccc23)NC(=O)[C@H](CC(C)C)NC(=O)[C@H](CO)NC(=O)CNC(=O)[C@H]([C@@H](C)CC)NC(=O)[C@H]([C@@H](C)CC)NC1=O]

In preferred embodiments, the compound of general formula (I) is cyclic peptide 2 or a salt thereof:

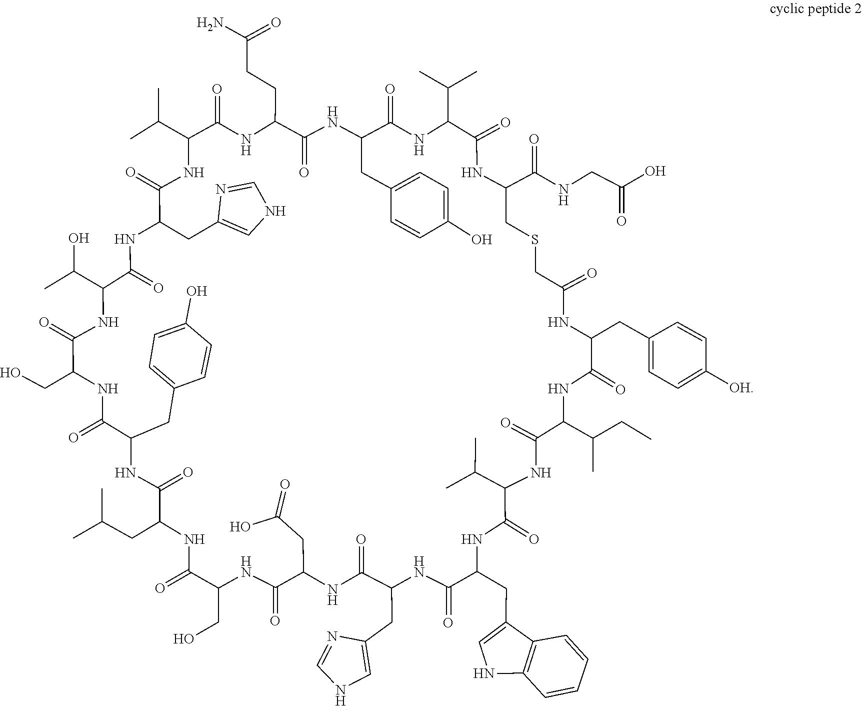

A preferred cyclic peptide 2 is cyclic peptide 2L:

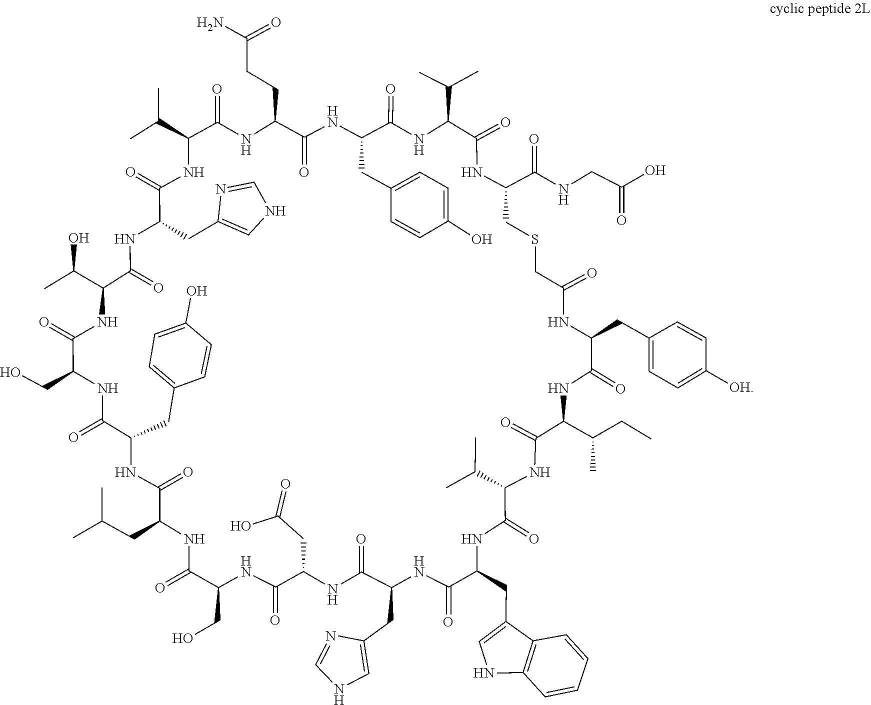 [SMILES: CC[C@H](C)[C@@H]1NC(=O)[C@H](Cc2ccc(O)cc2)NC(=O)CSC[C@@H](C(=O)NCC(=O)O)NC(=O)[C@H](C(C)C)NC(=O)[C@H](Cc2ccc(O)cc2)NC(=O)[C@H](CCC(N)=O)NC(=O)[C@H](C(C)C)NC(=O)[C@H](Cc2c[nH]cn2)NC(=O)[C@H]([C@@H](C)O)NC(=O)[C@H](CO)NC(=O)[C@H](Cc2ccc(O)cc2)NC(=O)[C@H](CC(C)C)NC(=O)[C@H](CO)NC(=O)[C@H](CC(=O)O)NC(=O)[C@H](Cc2c[nH]cn2)NC(=O)[C@H](Cc2c[nH]c3ccccc23)NC(=O)[C@H](C(C)C)NC1=O]

### Composition

The invention provides a composition comprising at least one compound of general formula I, and a pharmaceutically acceptable excipient, preferably for use according to the invention (use is described elsewhere herein). Such a composition is referred to herein as a composition according to the invention. Preferred compositions according to the invention are pharmaceutical compositions. In preferred embodiments, the composition according to the invention is formulated for oral, sublingual, parenteral, intravascular, intravenous, subcutaneous, or transdermal administration, optionally for administration by inhalation; preferably for oral administration. More features and definitions of administration methods are provided in the section on formulation and administration.

### Formulation and Administration

The compositions comprising the compounds as described above, can be prepared as a medicinal preparation or in various other media, such as foods for humans or animals, including medical foods and dietary supplements. The compounds and compositions may be compounded with other physiologically acceptable materials that can be ingested including, but not limited to, foods. In addition, or alternatively, the compositions as described herein may be administered orally in combination with (the separate) administration of food.

The compositions or compound according to the invention may be administered alone or in combination with other pharmaceutical agents and can be combined with a physiologically acceptable carrier thereof. In particular, the compounds described herein can be formulated as pharmaceutical compositions by formulation with additives such as pharmaceutically or physiologically acceptable excipients carriers, and vehicles. Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, and the like, as well as combinations of any two or more thereof. Other suitable pharmaceutically acceptable excipients are described in “Remington's Pharmaceutical Sciences, “Mack Pub. Co., New Jersey (1991), and “Remington: The Science and Practice of Pharmacy,” Lippincott Williams & Wilkins, Philadelphia, 20th edition (2003), 21^{st} edition (2005) and 22^{nd} edition (2012), incorporated herein by reference.

Compositions for use according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in a conventional manner using one or more physiologically acceptable carriers comprising excipients and auxiliaries that facilitate processing of the active compounds into preparations which can be used pharmaceutically. Proper formulation is dependent on the route of administration chosen.

For injection, the compounds and compositions for use according to the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Oral and parenteral administration may be used where the compounds and compositions for use are formulated by combining them with pharmaceutically acceptable carriers well known in the art, or by using them as a food additive. Such strategies enable the compounds and compositions for use according to the invention to be formulated as tablets, pills, dragées, capsules, liquids, gels, syrups, slurries, suspensions and the like, for oral ingestion by a subject to be treated. Preparations or pharmacological preparations for oral use may be made with the use of a solid excipient, optionally grinding the resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate. Additionally, coformulations may be made with uptake enhancers known in the art.

Compounds and compositions which can be administered orally include push-fit capsules made of gelatin, as well as soft, sealed capsules made of gelatin and a plasticizer, such as glycerol or sorbitol. The push-fit capsules may contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compounds and compositions for use according to the invention may be administered in the form of tablets or lozenges formulated in a conventional manner.

The compounds and compositions for use according to the invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. This formulation is preferred because it enables specific targeting of muscle tissue.

Compositions for parenteral administration include aqueous solutions of the compositions in water-soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, one or more components of the composition may be in powder form for constitution with a suitable vehicle, e.g., sterile pyrogen-free water, before use.

The compositions for use according to the invention also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Compositions for use according to the invention include compounds and compositions wherein the active ingredients are contained in an amount effective to achieve their intended purposes. More specifically, a therapeutically effective amount means an amount of compound effective to prevent, stabilize, alleviate, revert, or ameliorate causes or symptoms of disease, or prolong the survival, mobility, or independence of the subject being treated. Determination of a therapeutically effective amount is within the capability of those skilled in the art, especially in light of the disclosure provided here. For any compounds and compositions used in the invention, the therapeutically effective amount or dose can be estimated initially from cell culture assays, for example, as exemplified herein. Dosage may vary within this range depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics” Ch. 1 p. 1). The amount of compound and compositions administered will, of course, be dependent on the subject being treated, the subject's weight, the severity of the affliction, the manner of administration and the judgment of the prescribing physician.

An “effective amount” of a compound or composition is an amount which, when administered to a subject, is sufficient to reduce or eliminate either one or more symptoms of a disease or to retard the progression of one or more symptoms of a disease, or to reduce the severity of one or more symptoms of a disease, or to suppress the manifestation of a disease, or to suppress the manifestation of adverse symptoms of a disease. An effective amount can be given in one or more administrations.

The “effective amount” may be combined with the carrier materials to produce a single dosage form will vary depending upon the host to which the active ingredient is administered and the particular mode of administration. The unit dosage chosen is usually fabricated and administered to provide a desired final concentration of the compound in the blood.

The effective amount (i.e. the effective total daily dose), preferably for adults, is herein defined as a total daily dose of about 0.01 to 2000 mg, or about 0.01 to 1000 mg, or about 0.01 to 500 mg, or about 5 to 1000 mg, or about 20 to 800 mg, or about 30 to 800 mg or about 30 to 700 mg, or about 20 to 700 mg or about 20 to 600 mg, or about 30 to 600 mg, or about 30 to 500 mg, about 30 to 450 mg or about 30 to 400 mg, or about 30 to 350 mg or about 30 to 300 mg or about 50 to 600 mg, or about 50 to 500 mg, or about 50 to 450 mg, or about 50 to 400 mg or about 50 to 300 mg, or about 50 to 250 mg, or about 100 to 250 mg or about 150 to 250 mg. In the most preferred embodiment, the effective amount is about 200 mg. In preferred embodiments, the invention provides a compound for use according to the invention, or a composition for use according to the invention, characterized in that it is administered to a subject in an amount ranging from 0.1 to 1500 mg/day, preferably from 0.1 to 1000 mg/day, more preferably from 0.1 to 400 mg/day, still more preferably from 0.25 to 150 mg/day, such as about 100 mg/day.

Alternatively, the effective amount of the compound, preferably for adults, preferably is administered per kg body weight. The total daily dose, preferably for adults, is therefore about 0.05 to about 40 mg/kg, about 0.1 to about 20 mg/kg, about 0.2 mg/kg to about 15 mg/kg, or about 0.3 mg/kg to about 15 mg/kg or about 0.4 mg/kg to about 15 mg/kg or about 0.5 mg/kg to about 14 mg/kg or about 0.3 mg/kg to about 14 mg/kg or about 0.3 mg/kg to about 13 mg/kg or about 0.5 mg/kg to about 13 mg/kg or about 0.5 mg/kg to about 11 mg/kg.

Alternative examples of dosages which can be used are an effective amount of the compounds for use according to the invention within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight.

In a preferred embodiment of the invention, “subject”, “individual”, or “patient” is understood to be an individual organism, preferably a vertebrate, more preferably a mammal, even more preferably a primate and most preferably a human.

### Use

The invention provides a compound according to the invention, or a composition according to the invention, for use as a medicament. The medicament can be for treating, preventing, or ameliorating a coronaviral infection. The viral infection is preferably an infection by a respiratory virus, such as RSV, a pneumovirus, an influenza virus or a coronavirus. More preferably, the viral infection is an infection by a coronavirus, such as SARS-CoV or SARS-CoV-1, MERS-CoV or SARS-CoV-2. Most preferably, the viral infection is an infection by SARS-CoV-2, i.e. is COVID-19. Methods for administration are described above.

A preferred coronaviral infection is a SARS-CoV-2 infection. Preferably the medicament is for treating a viral infection, preferably a coronaviral infection, more preferably a SARS-CoV-2 infection. Treatment is preferably prophylactic treatment. The inhibition of viral spike protein binding reduces infectivity of the virus, ensuring effective prophylactic treatment. In some embodiments the medicament is for treating, ameliorating, or preventing COVID-19.

Also, the use is provided of either a compound of general formula I, or of a composition according to the invention, for the treatment, prevention, or amelioration of a viral infection in a subject in need thereof, and comprises administration to the subject of an effective dose of a compound of general formula I or composition according to the invention.

### Method

The invention provides an in vitro, in vivo, or ex vivo method for modulating the conformational dynamics of a viral spike protein, wherein the method comprises the step of contacting the viral spike protein with a compound according to the invention, or with a composition according to the invention. In some embodiments the method is in vitro or ex vivo. In some embodiments the method is in vitro.

Preferably the method is for modulating the conformational dynamics of a viral spike protein of a coronaviral particle, more preferably of a SARS-CoV-2 particle. Preferably, the modulation prevents interaction between the spike protein and an ACE2 receptor. In some embodiments, the modulation is for preventing the opening of a spike RBD. Preferably, the modulation is for stabilizing the closed conformation of a spike RBD, more preferably of a SARS-CoV-2 spike protein RBD.

The method preferably comprises use as defined earlier herein. The viral spike protein can be inside a cell, in a cell culture medium, or it can be in a sample or subject. The viral spike protein is preferably comprised in a viral particle. Preferred methods comprise contacting a cell, sample, or subject with a compound of general formula I or composition as defined earlier herein. In the context of the invention, contacting a cell, sample, or subject with a compound of general formula I or a composition can comprise adding such a compound of general formula I or composition to a medium in which a cell is cultured. Contacting a cell with a compound of general formula I or a composition can also comprise adding such a compound of general formula I or composition to a medium, buffer, or solution in which a cell is suspended, or which covers a cell, or to with which a sample was admixed. Further methods for administration are defined elsewhere herein. The cell may be a cell from a sample obtained from a subject. Such a sample may be a sample that has been previously obtained from a subject. Within the embodiments of this aspect, samples may have been previously obtained from a human subject. Within the embodiments of this aspect, samples may have been obtained from a non-human subject. In a preferred embodiment of this aspect, obtaining the sample is not part of the method according to the invention.

The invention also provides a method of treating a viral infection, the method comprising administering to a subject a compound according to the invention, or a composition according to the invention. Further features and definitions are as provided elsewhere herein.

### General Definitions

When a structural formula or chemical name is understood by the skilled person to have chiral centers, yet no chirality is indicated, for each chiral center individual reference is made to all three of either the racemic mixture, the pure R enantiomer, and the pure S enantiomer. Whenever a fragment of a molecule, often referred to as a moiety, is represented, a dotted or wavy line indicates which bond links it to the entirety of the molecule; alternately, an asterisk (*) indicates where the represented moiety is linked to the rest of the molecule. This asterisk does not imply an atom, and neither does a bond that is crossed by a dotted or wavy line convey information about which atom is at the non-moiety side of the bond.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used as a medicament, it can also be used for the manufacture of a medicament, or in a method. In preferred embodiments, compounds and compositions according to the invention are for use in methods according to the invention, or are for use according to the invention.

In this document and in its claims, the verb “to comprise” and its conjugations are used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 5%, preferably 1% of the value.

All patent and literature references cited in the present specification are hereby incorporated by reference in their entirety.

### EXAMPLES

### 1. Provision of Compounds

Peptides were synthesized using micro-wave assisted Fmoc solid phase peptide synthesis on Rapp Polymere (Germany) tentagel S RAM resin on a Liberty Blue (CEM) Peptide Synthesizer. Each coupling step of 4 minutes was performed with 5 equivalents of amino acid, 5 equivalents of Oxyma Pure and 10 equivalents of N,N′-Diisopropylcarbodiimide (DIC) in DMF at 90° C. Histidine coupling was performed at 50° C. for 10 minutes instead. Fmoc deprotection was performed using 20% piperidine in DMF for 1 min at 90° C. Automated synthesis was followed by N-terminal chloroacetylation by using 5 equivalents of chloroacetic acid instead of amino acid in the described coupling step, or N-acetylation by treatment with 20% acetic anhydride in DMF for 2 minutes at 65° C. Peptide cleavage, global deprotection, cyclization and subsequent purification were performed by addition of cleavage solution (90% trifluoroacetic acid, 5% water, 2.5% triisopropyl silane, 2.5% 1,2-ethanedithiol) and incubating with gentle shaking for 2-3 hours. Resin was filtered off and the cleaved deprotected peptide was precipitated by addition to a 10-fold excess of cold diethyl ether. The peptide was pelleted by centrifugation at 5,000 rcf for 5 min and the supernatant removed. A further portion of diethyl ether was added and the pellet resuspended before again centrifuging and removing supernatant. The pellet was washed in this way a total of three times, then allowed to dry in the air before dissolving to approximately 5 mM in dimethylsulfoxide. A few drops of DIPEA base was then added and the cyclization reaction was allowed to proceed overnight before quenching with an equal volume of trifluoroacetic acid. The resulting solution was loaded directly onto a C18 column for HPLC purification with an acetonitrile gradient from 10-70% in water. Purified peptides were characterized by HPLC for purity and coupled to mass spectrometry for identification.

### 2. Characterization of Compounds

### 2.1 Physical Characterization

Shown below is mass spectrometry data, both calculated and observed, for the provided compounds (linker is L4a; X is NH; as for cyclic peptide 1L; except S1b3inL1lin and SARS2L1lin, which are Ac-[sequence of SEQ ID NO]-NH₂). Peptides were sufficiently pure by HPLC analysis.

TABLE S2.1
mass spectrometry of certain compounds, M + 2H

| | | | SEQ |
|---|---|---|---|
| Compound | Calc. | Obs. | ID NO |
| SARS2D1 | 1113.6 | 1114.3 | 3 |
| SARS2D4 | 1101.1 | 1101.4 | 4 |
| SARS2L1 | 1105.0 | 1105.3 | 2 |
| SARS2L2 | 1106.0 | 1106.4 | 5 |
| SARS2L3 | 1076.0 | 1076.2 | 6 |
| SARS2R3L1 | 1052.5 | 1052.8 | 7 |
| SARS2R3L2 | 1028.0 | 1028.3 | 8 |
| S1b3inL1 | 1116.6 | 1116.9 | 1 |
| S1b3inL4 | 1073.6 | 1073.9 | 9 |
| SARS1spikeL1 | 1194.1 | n.o. | 10 |
| SARS1spikeL2 | 1221.1 | 1221.4 | 11 |
| SARS1spikeL3 | 1142.0 | n.o. | 12 |
| SARS1spikeD2 | 1011.5 | 1011.8 | 13 |
| S1b3inL1lin | 1101.6 | 1101.9 | 31 |
| SARS2L1lin | 1090.0 | 1090.3 | 32 |
| S1b3inL1scr | 1116.6 | 1117.2 | 14 |
| SARS2L1scr | 1105.0 | 1105.6 | 15 |
| Pos1 | 1074.1 | 1074.4 | 16 |
| Pos2 | 1074.1 | 1074.4 | 17 |
| Pos3 | 1103.6 | 1104.2 | 18 |
| Pos4 | 1074.1 | 1074.8 | 19 |
| Pos5 | 1087.6 | 1088.3 | 20 |
| Pos6 | 1088.1 | 1088.4 | 21 |
| Pos7 | 1095.6 | 1096.2 | 22 |
| Pos8 | 1095.6 | 1096.2 | 23 |
| Pos9 | 1095.6 | 1096.3 | 24 |
| Pos10 | 1123.6 | 1124.3 | 25 |
| Pos11 | 1108.6 | 1108.9 | 26 |
| Pos12 | 1095.6 | 1096.2 | 27 |
| Pos13 | 1059.2 | 1059.4 | 28 |
| Pos14 | 1102.6 | 1102.9 | 29 |
| Pos15 | 1078.6 | 1079.3 | 30 |

### 2.2 Spike Protein Binding

Compounds that bind to the spike protein of the SARS-CoV-2 virion were identified by mRNA display of compounds, using affinity panning with immobilized spike protein over 8 sequential rounds of affinity enrichment followed by library regeneration as described in van Haren et al., RSC Chem. Biol. 2, 1546-1555, 2021. Further panning of the enriched library resulting from the third round of the above selection was carried out for two rounds with the isolated S1b domain of the spike protein. All enriched libraries were subjected to high throughput sequencing and sequences of binding compounds determined by analysis of highly enriched unique sequences. The level of enrichment for selected compounds is summarized in the following table, each enriched from an estimated starting fraction of 1 in 1012 sequences (0.0000000001%).

TABLE S2.2
enrichment in final round (% of total
sequences) of certain compounds

| Compound | Enrichment |
|---|---|
| SARS2D1 | 11.88% |
| SARS2D2 | 11.51% |
| SARS2D3 | 5.68% |
| SARS2D4 | 5.53% |
| SARS2L1 | 17.89% |
| SARS2L2 | 17.02% |
| SARS2L3 | 1.25% |
| SARS2R3L1 | 0.71% |
| SARS2R3L2 | 0.77% |
| S1b D1 | 4.48% |
| S1b3inD1 | 5.05% |
| S1b3inD13 | 0.87% |
| S1b3inD22 | 0.44% |
| S1b3inD31 | 0.35% |
| S1b3inL1 | 2.15% |
| S1b3inL4 | 1.50% |
| SARS1spikeL1 | 8.32% |
| SARS1spikeL2 | 5.58% |
| SARS1spikeL3 | 2.90% |
| SARS1spikeD2 | 7.71% |

Binding of a subset of these identified compounds to the spike protein was further assessed by a thermal shift assay, with the magnitude of protein melting point change summarized in the following table. In this assay, purified compounds were prepared as a 400 μM stock solution in DMSO, based on UV absorbance at 280 nm with calculated extinction coefficients (Expasy ProtParam, Swiss Bioinformatics Resource Portal). Thermal shift assay reaction mixtures for measurements with full length SARS-CoV-2 Spike contained 10 μM compound, 0.25 mg/ml protein and 5× SYPRO orange dye (Thermo Fisher Scientific) in Dulbecco's phosphate-buffered saline (Gibco). Data collected for the S1b3inL1 compound had an altered concentration of 0.1 mg/ml full length SARS-CoV-2 spike. An equal volume of DMSO compared to the compound solution was added for the control experiments, leading to a final concentration of 5% DMSO. Assays were performed on a Bio-Rad CFX96 PCR machine using the ‘melting assay’ protocol (FRET filter settings) with increment steps of 0.5° C. for 10 seconds from 20 to 75° C. All measurements were performed in duplicate and averaged. Temperature corresponding to the highest value of the d (RFU)/dT peak was used to determine the melting temperature (Tm).

TABLE S2.2.2
melting point change for certain compounds

| Compound | ΔTm (° C.) |
|---|---|
| SARS2L1 | 2 |
| SARS2L2 | 2 |
| SARS2R3L2 | 1.5 |
| SARS2R3L1 | 1 |
| SARS2D3 | 1 |
| S1b3inL1 | 1 |
| S1b3inL4 | 0 |
| SARS2D1 | 0 |
| SARS2D2 | 0 |
| SARS2L3 | 0 |
| S1b3inD1 | 0 |
| S1b3inD13 | 0 |
| S1b3inD22 | 0 |
| S1b3inD31 | −0.5 |
| SARS2D4 | −0.5 |

### 2.3 Protective Binding

Of the above compounds found to bind to the spike protein, several were observed to give a protective effect in cellular assays of SARS2-S pseudotyped VSV infection (Wang et al., Nat. Comm. 11, 2251, 2020) and subsequently for a subset of these compounds with whole SARS-CoV-2 virus using the R-20 platform (Aggarwal et al., Nat. Mol. Biol. 7, 896-908, 2022). This testing is summarized in the table below.

TABLE S2.3
virus binding for certain compounds

| Compound | Pseudovirus IC₅₀ (uM) | Whole virus IC₅₀ (uM) |
|---|---|---|
| SARS2L1 | 0.41 | >100 |
| S1b3inL1 | 0.81 | 5.2 |
| S1b3inL4 | n.d. | 11.4 |

Of these inhibitory compounds, the most promising compound, comprising SEQ ID NO: 1, was further shown to be protective in cellular assays with SARS2-S pseudotyped VSV infection derived from diverse variants of concern. This indicates an unusually broad range of activity for a spike-binding molecule. Summarized in the table below is the half maximal inhibitor effect (IC₅₀) for this compound in these assays.

TABLE S2.3.2
further virus binding for certain compounds

| | Pseudovirus IC₅₀ (uM) | | | | |
|---|---|---|---|---|---|
| Compound | Alpha | Beta | Delta | Omicron BA.1 | Omicron BA.2 |
| S1b3inL1 | 2.6 | 5.2 | 3.5 | 5.2 | 3.5 |

This compound was further shown to be protective in cellular assays of infection with VSV pseudotyped with spike protein from several different sarbecoviruses. This indicates that the site of binding and mechanism of action is highly conserved. Summarized in the table below is the half maximal inhibitor effect (IC₅₀) for this compound in these assays.

TABLE S2.3.3
further virus binding for certain compounds

Pseudovirus IC₅₀ (uM)

| Compound | SARS-CoV-2 | SARS-CoV-1 | WIV-16 |
|---|---|---|---|
| S1b3inL1 | 1.7 | 2.2 | 7 |

### 2.4 Dimerization Further Increases Potency

Dimeric compounds were synthesized as described for the other compounds above, but using a lysine residue as a branch point early in the synthesis. This was implemented using a building block N,N′-Bis-Fmoc-L-lysine, wherein both the backbone and side-chain amine groups can be liberated on treatment with piperidine during the standard deprotection cycle. Both arms of the dimer were then extended in the same reaction for each reaction cycle. Reagent equivalents were calculated based on the dimeric compound, meaning that equivalents were doubled for amino acids incorporated before this branching. Compounds with different dimerizing linkers were produced (peptide is SEQ ID NO: 1; linker is DiL4; X is NH; see table S2.4). Characterization of the resulting dimeric compound by mass spectrometry is presented in the following table. All compounds were found to be sufficiently pure by HPLC analysis.

TABLE S2.4
characteristics for certain compounds

| | | Calc. | Calc. | Calc. | Calc. |
|---|---|---|---|---|---|
| | Dimerizing | M + 7H | M + 6H | M + 5H | M + 4H |
| Dimer | linker | (obs.) | (obs.) | (obs.) | (obs.) |
| Dimer 2 | DL1 | 745.4 (745.9) | 869.5 (870.0) | 1043.1 (1043.7) | 1303.7 (1304.0) |
| Dimer 1 | DL5 | 828.3 (828.8) | 966.2 (966.7) | 1159.2 (1159.8) | 1448.8 (1449.5) |
| Dimer 3 | DL7 | 911.2 (911.8) | 1062.9 (1063.5) | 1275.3 (1276) | 1593.8 (1594.5) |
| Dimer 4 | DL8 | 994.1 (994.7) | 1159.6 (1160.3) | 1391.3 (1392.1) | 1738.9 (1739.8) |

The resulting dimeric macrocyclic compounds were tested for inhibition of SARS-CoV-2 infection using pseudoviruses in a cellular assay, with activity summarized in the following table.

TABLE S2.4.1
virus binding for certain compounds

| Compound | IC₅₀ (uM) |
|---|---|
| S1b3inL1 | 1.5 |
| Dimer 2 | 0.05 |
| Dimer 1 | 0.03 |
| Dimer 3 | 0.09 |
| Dimer 4 | 0.01 |

These data show that the dimerization results in an increase in activity, and that this increased activity does not strongly depend on the linker used.

### 3. Robustness of the Compounds

Within compounds where linker is L4a, and X is NH (as for cyclic peptide 1L) each amino acid position in the peptide of SEQ ID NO: 1 was systematically replaced with alanine. Infection inhibition was measured for the resulting compounds in a pseudovirus cellular infection assay, with the data summarized in the table below. In this table, “n.i.” indicates no inhibition was observed.

TABLE S3
infection inhibition for certain compounds

| SEQ ID NO: | Alanine at . . . | IC₅₀ (uM) |
|---|---|---|
| 1 | No mutation | 0.09 |
| 16 | 1 | n.i. |
| 17 | 2 | 34.68 |
| 18 | 3 | 23.03 |
| 19 | 4 | 1.07 |
| 20 | 5 | 4.40 |
| 21 | 6 | 1.98 |
| 22 | 7 | n.i. |
| 23 | 8 | 72.14 |
| 24 | 9 | 11.42 |
| 25 | 10 | 54.76 |
| 26 | 11 | 56.94 |
| 27 | 12 | n.i. |
| 28 | 13 | 314.9 |
| 29 | 14 | 0.23 |
| 30 | 15 | 0.47 |

For many of the compounds in the table above some degree of activity was retained, and this degree of activity was taken as an indication of the importance of the original residue at that position. Compounds comprising peptides with mutations at positions 1, 7, and 12 showed no inhibition, and so these positions are particularly relevant for activity. Peptides with mutations at positions 2, 3, 8, 10, 11, and 13, and to some extent 9, showed attenuated inhibition, and so these positions are seen as contributing to activity. Peptides with mutations at positions 4, 5, and 6 showed some loss of inhibition, and so these positions are seen as somewhat contributing to activity. Peptides with mutations at positions 14 and 15 showed only a small loss in inhibition, and so these positions are seen as unimportant for activity. These results demonstrate that the compounds according to the invention are resistant to changes in peptide without loss of activity.

Many more mutants were then assayed, replacing positions by amino acids other than alanine. Double mutations were well-tolerated. For single mutations, results are shown in Table S4, where one letter amino acid codes (along with X and Z as defined earlier herein) are used and the top line denotes the position in SEQ ID NO: 1 from N to C terminus. The column with mutations designates which mutations were made at the indicated position. Table S4 shows binding affinity standardized to that of SEQ ID NO: 1, which represents 100% binding. Results labeled “−” represent decreased binding. Results labeled “0” represent substantially similar binding. Results labeled “1” represent the original sequence. Results labeled “+” represent about 105-200% binding. Results labeled “++” represent about 200-300% binding. Results labeled “+++” represent about 300-500% binding. Results labeled “++++” represent over about 500% binding.

TABLE S4
binding for certain compounds, standardized to SEQ ID NO: 1

| Mutation | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| G | − | − | − | − | − | − | − | − | − | 1 | − | − | − | +++ | − |
| A | − | − | + | 0 | + | + | − | − | − | − | − | − | − | ++ | + |
| V | − | − | − | − | − | − | +++ | − | − | − | − | ++++ | − | 1 | + |
| I | − | − | − | − | − | 0 | 1 | 1 | 1 | − | − | +++ | − | − | +++ |
| L | − | − | ++ | − | − | − | − | − | − | − | − | 1 | − | − | +++ |
| F | − | − | − | − | 0 | − | − | − | 0 | − | − | − | − | − | 1 |
| Z | 0 | 0 | + | + | − | − | − | − | ++ | − | − | ++ | − | − | 0 |
| Y | − | − | − | − | + | + | − | − | − | − | − | − | − | + | − |
| X | − | − | 0 | − | +++ | ++ | − | − | − | − | − | − | − | 0 | − |
| W | − | − | − | − | +++ | ++ | − | − | + | − | − | − | 1 | − | − |
| S | − | − | + | − | + | 0 | − | − | − | − | 1 | − | − | ++ | − |
| T | − | − | + | − | − | + | − | − | − | − | − | − | − | ++ | − |
| N | − | − | + | − | + | − | − | − | − | − | − | − | − | − | − |
| Q | + | − | 0 | − | − | 1 | − | − | − | − | − | − | − | ++ | ++ |
| H | − | − | 0 | − | + | + | − | − | − | − | − | − | − | +++ | + |
| K | + | − | + | − | − | − | − | − | − | − | − | − | − | − | − |
| R | 1 | 1 | + | 1 | − | − | − | − | − | − | − | + | − | − | + |
| D | − | − | − | − | + | 0 | − | − | − | − | − | − | − | +++ | − |
| E | − | − | − | − | 1 | ++ | − | − | − | − | − | − | − | ++++ | ++ |
| P | − | − | 1 | + | − | − | − | − | − | − | − | − | − | − | + |
| C | + | +++ | +++ | ++ | +++ | +++ | 0 | − | − | − | + | − | − | ++ | + |

1. Compound of general formula (I): 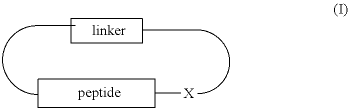
wherein
peptide is an oligopeptide having 13-17 amino acid residues, preferably comprising the oligopeptide represented by SEQ ID NO: 1 having at most 5 amino acid substitutions;
linker is a linking moiety that together with X connects the N-terminus and the C-terminus of peptide to form a macrocyclic structure; and
X is O or NH,
or a salt of such a compound.

2. The compound according to claim 1, wherein peptide is an oligopeptide having 13-17 amino acid residues, wherein
the first amino acid is arginine, lysine, histidine, leucine, isoleucine, or valine;
the seventh amino acid is isoleucine, leucine, or valine; and
the twelfth amino acid is isoleucine, leucine, or valine.

3. The compound according to claim 1, wherein peptide has a length of 15-17, preferably 15 amino acids and is an oligopeptide represented by SEQ ID NO: 1 having 5, 4, 3, 2, or 1 amino acid substitutions.

4. The compound according to claim 3, wherein within peptide when position 1 is substituted by another amino acid, it is preferably substituted by Z, Q, L, or C, more preferably by Q, K, or C, most preferably K; when position 2 is substituted by another amino acid, it is preferably substituted by Z or C, more preferably by C; when position 3 is substituted by another amino acid, it is preferably substituted by A, L, Z, X, S, T, N, Q, H, L, R, or C, more preferably by L, S, T, N, K, R, or C, most preferably by C; when position 4 is substituted by another amino acid, it is preferably substituted by A, Z, P, or C, more preferably by Z, P, or C, most preferably by C; when position 5 is substituted by another amino acid, it is preferably substituted by A, F, Y, X, W, S, N, H, R, D, or C, more preferably by A, Y, X, W, S, N, H, or C, most preferably by X, W, or C; when position 6 is substituted by another amino acid, it is preferably substituted by A, I, Y, X, W, S, T, H, D, E, or C, more preferably by X, W, E, or C, most preferably by C; when position 7 is substituted by another amino acid, it is preferably substituted by V; position 8 is not substituted; when position 9 is substituted by another amino acid, it is preferably substituted by F, Z, or W, more preferably by Z or W, most preferably by Z; when position 10 is substituted by another amino acid, it is preferably substituted by C; when position 11 is substituted by another amino acid, it is preferably substituted by C; when position 12 is substituted by another amino acid, it is preferably substituted by V, I, Z, T, R, or C, more preferably by V, I, Z, or R, most preferably by V or I; position 13 is not substituted; when position 14 is substituted by another amino acid, it is preferably substituted by G, A, Y, X, S, T, Q, H, D, E, or C, more preferably by G, A, S, T, Q, H, D, E, or C, even more preferably by G, H, D, or E, most preferably by E; when position 15 is substituted by another amino acid, it is preferably substituted by A, V, I, L, Z, Q, H, R, E, P, or C, more preferably by A, V, I, L, Q, E, or P, even more preferably by I, L, Q, or E, most preferably by I or L; wherein a substitution by X denotes a substitution by 3,4-di-hydroxy-phenylalanine and a substitution by Z denotes a substitution by pentafluorophenylalanine.

5. The compound according to claim 4, wherein within peptide substitutions are made only on positions 2, 3, 4, 5, 6, 7, 9, 12, 14, or 15, preferably only on positions 2, 3, 5, 6, 7, 12, 14, or 15, still more preferably only on positions 5, 12, or 14, most preferably only on positions 12 or 14.

6. The compound according to claim 1, wherein linker comprises 1 to 12 optionally substituted backbone atoms selected from carbon, nitrogen, oxygen, and sulphur,
wherein optional substitutions can be —O, halogen, C1-4 hydrocarbon, C1-4 acyl, C1-4 alkoxy, —C(═O)-oligopeptide, —SH, —S—(C1-4 hydrocarbon), —NH₂, —NH—(C1-4 hydrocarbon), —NH—(C1-4 acyl), —N—(C1-4 hydrocarbon)₂, —N—(C1-4 acyl)₂, an amino acid side chain, or a targeting moiety.

7. The compound according to claim 1, wherein linker has general formula (L1): 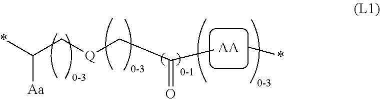
wherein
Aa is H, —NH₂, —COOH, —CONH₂, -(AA)ₙ-X′, —C(═O)(AA)ₙ-X′, or a dimerizing linker;
Q is CH₂, O, S, or NH; and
AA is in each instance an independently selected amino acid residue;
X′ is OH or NH₂; and
n is 1, 2, 3, 4, 5, 6, 7, 8, 9, or 10.

8. The compound according to claim 1, wherein linker has general formula (L2): 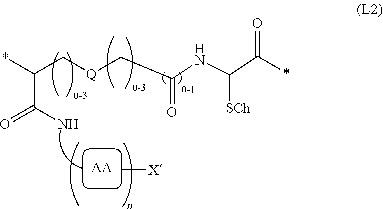
wherein
X′ is OH or NH₂;
Q is CH₂, O, S, or NH;
SCh is an amino acid side chain;
AA is in each instance an independently selected amino acid residue; and
n is 1, 2, 3, 4, or 5.

9. The compound according to claim 1, wherein linker has general formula (L3): 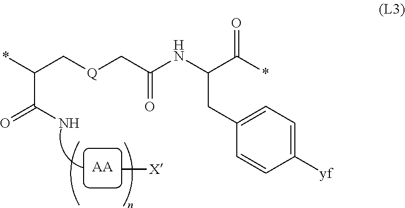
wherein
X′ is OH or NH₂;
Q is CH₂, O, S, or NH;
yf is H or OH;
AA is in each instance an independently selected amino acid residue; and
n is 1, 2, or 3.

10. The compound according to claim 1, wherein linker has a general formula selected from (L4a) through (L4h):

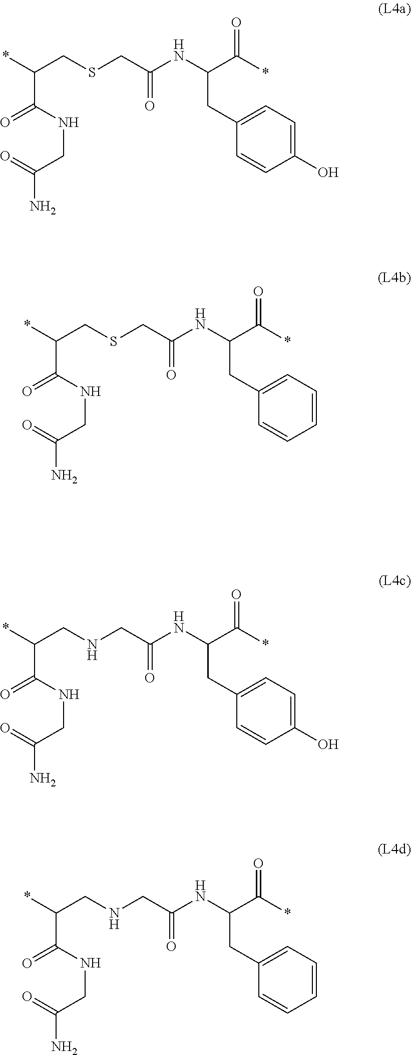

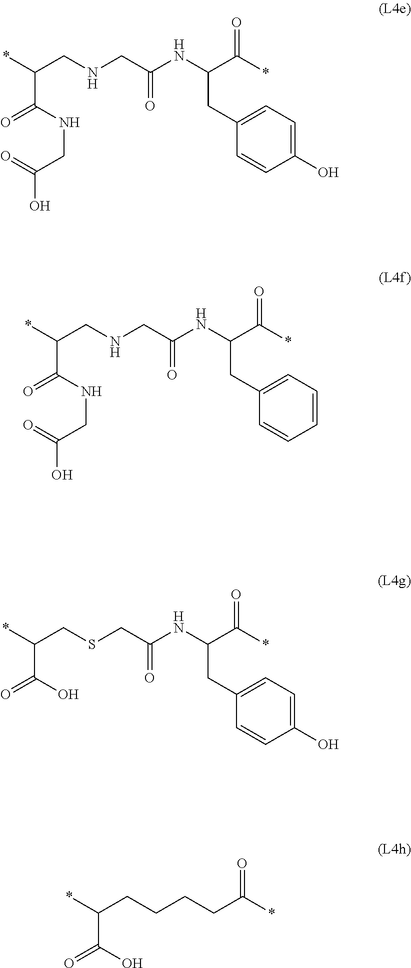

11. The compound according to claim 1, wherein peptide is an oligopeptide having 13-17 amino acid residues, wherein
the first amino acid is arginine, histidine, or lysine, preferably arginine;
the seventh amino acid is isoleucine or leucine, preferably isoleucine;
the twelfth amino acid is isoleucine or leucine, preferably leucine.

12. The compound according to claim 1, wherein
peptide comprises a sequence represented by any one of SEQ ID NOs: 1-30, wherein up to six positions can be substituted by another amino acid.

13. The compound according to claim 1, wherein
peptide has 15 amino acid residues; and/or
peptide comprises a sequence represented by any one of SEQ ID NOs: 1-2; and/or
linker has any one of general formulas (L4a) through (L4h); and/or
X is NH.

14. The compound according to claim 1, wherein it is cyclic peptide 1 or a salt thereof:

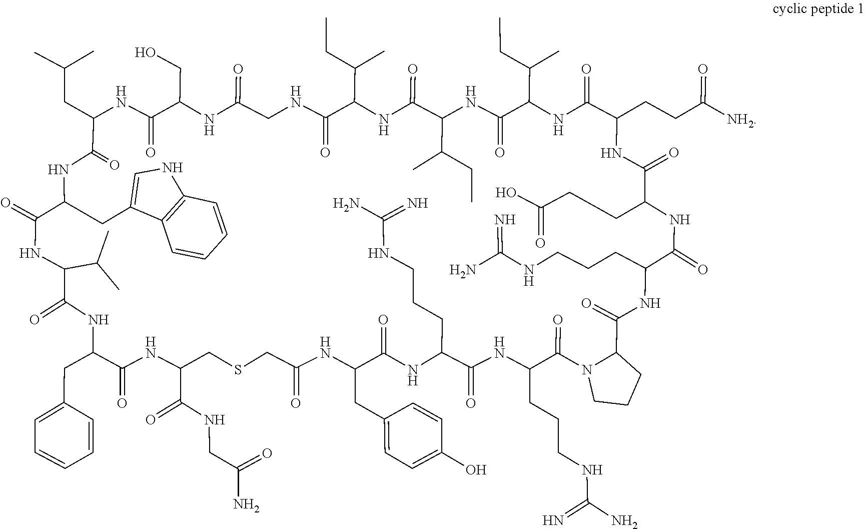 [SMILES: CCC(C)C1NC(=O)C(CCC(N)=O)NC(=O)C(CCC(=O)O)NC(=O)C(CCCNC(=N)N)NC(=O)C2CCCN2C(=O)C(CCCNC(=N)N)NC(=O)C(CCCNC(=N)N)NC(=O)C(Cc2ccc(O)cc2)NC(=O)CSCC(C(=O)NCC(N)=O)NC(=O)C(Cc2ccccc2)NC(=O)C(C(C)C)NC(=O)C(Cc2c[nH]c3ccccc23)NC(=O)C(CC(C)C)NC(=O)C(CO)NC(=O)CNC(=O)C(C(C)CC)NC(=O)C(C(C)CC)NC1=O]

15. The compound according to claim 1, wherein peptide is an oligopeptide represented by any one of SEQ ID NOs: 1, 33-102.

16. A composition comprising a compound as defined in claim 1 and a pharmaceutically acceptable excipient.

17. (canceled)

18. (canceled)

19. An in vitro, in vivo, or ex vivo method for modulating the conformational dynamics of a viral spike protein, wherein the method comprises the step of contacting the viral spike protein with a compound as defined in claim 1.

20. A method of treating a viral infection, the method comprising administering to a subject a compound as defined in claim 1.

21. The method of treatment according to claim 20, wherein the method is for treating a viral infection, preferably a coronaviral infection, more preferably a SARS-CoV-2 infection.
